(19) United States
(12) Patent Application Publication
(10) Pub. No.: US 2026/0115166 A1
(43) Pub. Date: Apr. 30, 2026

(54) COMPOUNDS AND METHODS OF USE THEREOF FOR THE TREATMENT OF PHOTORECEPTOR ROD DEGENERATIONS AND GENERATION OF PHOTORECEPTORS

(71) Applicant: Purdue Research Foundation, West Lafayette, IN (US)

(72) Inventor: Yuk Fai Leung, West Lafayette, IN (US)

(21) Appl. No.: 19/101,586

(22) PCT Filed: Aug. 5, 2023
(86) PCT No.: PCT/US2023/071751

§ 371 Date: Feb. 5, 2025

Related U.S. Application Data
(60) Provisional application No. 63/395,798, filed on Aug. 6, 2022.

(51) Int. Cl. A61K 31/403; A61K 35/30; A61K 45/06; A61P 27/02; C12N 5/0793

(52) U.S. Cl. CPC A61K 31/403; A61K 35/30; A61K 45/06; A61P 27/02; C12N 5/062

(57) ABSTRACT

Methods for facilitating the genesis of photoreceptor rods in a subject by administering a therapeutically effective amount of a therapeutic agent are provided. Other methods are provided for treating retinitis pigmentosa by administering a therapeutically effective dose of therapeutic agents. Methods for producing an in vitro population of progenitor cells and photoreceptor rods generated therefrom are also provided.

### PRIORITY

This application is related to and claims the priority benefit of U.S. Provisional Patent Application No. 63/395,798 filed Aug. 6, 2022. The content of the aforementioned application is hereby incorporated by reference in its entirety into this disclosure.

### TECHNICAL FIELD

The present disclosure relates to methods for facilitating regeneration or genesis of photoreceptor rods in a subject by administering a therapeutic agent. Methods for treating retinitis pigmentosa are also provided, as are methods for producing an in vitro population of progenitor cells and photoreceptor rods.

### BACKGROUND

Retinitis pigmentosa (RP) is a common group of genetically inherited retinal diseases that lead to blindness via retinal degeneration. RP affects approximately 1 in 4.000 people worldwide. Usually, patients first suffer from peripheral visual field loss because of peripheral rod photoreceptor cell degradation and eventually death caused by mutations in phototransduction genes including rhodopsin (RHO). Photoreceptor cell degradation and death gradually but steadily progresses until patients lose central visual function, which significantly degrades quality of life. Such slow progressive photoreceptor cell death is a prominent feature of RP. Additionally, there are numerous other retinal-degenerative diseases that result in the progressive loss of photoreceptor rods and eventually lead to blindness. When patients lose their vision, they suffer from increased likelihood of injury as well as increased anxiety and depression.

There are currently no effective treatment options available for the vast majority of patients suffering from these diseases (including RP). Research into technologies including gene therapy, stem-cell therapy, and retinal prosthesis are being explored; however, these options are experimental and cost-prohibitive. The only U.S. Food and Drug Administration-approved method for treating any form of RP is a recently developed gene therapy (Luxturna) for the treatment of Lebar's Congenital Amaurosis (LCA). Patients with biallelic RPE65 mutations preventing normal expression of the gene can be treated with Luxturna, which replaces the non-functional enzyme with a functional RPE65 via an adeno-associated virus. Maguire et al., Clinical Perspective: Treating RPE65-Associated Retinal Dystrophy, Molecular Therapies 29:442-463 (2021). While Luxturna has shown to be effective in restoring vision to some LCA patients, this is only a small portion of all patients suffering from rod degeneration diseases. Indeed, this treatment strategy (replacing a deficient enzyme) is not effective in other types of retinal-degenerative diseases (e.g., autosomal dominant cases of RP (adRP).

What is needed is an effective therapy for the treatment of RP (e.g., adRP) and other retinal-degenerative diseases. Additionally, there is a need for treatments that can promote the generation of new rod photoreceptors and/or regeneration of existing rod photoreceptors in mature patients.

### SUMMARY

Methods for photoreceptor rod regeneration or rod genesis (e.g., rod neogenesis) are provided. In certain embodiments, the method comprises administering, to a subject, a therapeutically effective dose of therapeutic agent selected from the group consisting of carvedilol, esmolol hydrochloric acid, triamterene, trelagliptin, prednisolone acetate, crenolanib, dolutegravir, tivantinib, noradrenaline bitartrate monohydrate, vidofludimus, gabapentin, gemcitabine hydrochloride (HCl), desvenlafaxine succinate. LCZ696 or sacubitril/valsartan. Palbociclib (PD0332991) isethionate, galanthamine hydrobromide (HBr), amitriptyline HCl, and xylazine HCl. In response to administration of the therapeutically effective dose of the therapeutic agent, the subject can experience enhanced regeneration or genesis of photoreceptor rods (e.g., depending on if the subject is mature or comprises progenitor cells (e.g., retinal progenitor cells)) as compared to a control subject that did not receive administration of the therapeutic agent. In other words, the therapeutically effective dose of the therapeutic agent can facilitate enhanced regeneration and/or genesis (or neogeneration) of new photoreceptor rods.

In certain embodiments, the therapeutic agent is carvedilol. In certain embodiments, the therapeutic agent is esmolol hydrochloric acid. In certain embodiments, the therapeutic agent is triamterene. In certain embodiments, the therapeutic agent is trelagliptin. In certain embodiments, the therapeutic agent is prednisolone acetate. In certain embodiments, the therapeutic agent is crenolanib. In certain embodiments, the therapeutic agent is dolutegravir. In certain embodiments, the therapeutic agent is tivantinib. In certain embodiments, the therapeutic agent is noradrenaline bitartrate monohydrate. In certain embodiments, the therapeutic agent is vidofludimus. In certain embodiments, the therapeutic agent is gemcitabine HCL. In certain embodiments, the therapeutic agent is desvenlafaxine succinate. In certain embodiments, the therapeutic agent is LCZ696 (sacubitril/valsartan). In certain embodiments, the therapeutic agent is Palbociclib (PD0332991) isethionate. In certain embodiments, the therapeutic agent is galanthamine HBr. In certain embodiments, the therapeutic agent is amitriptyline HCl. In certain embodiments, the therapeutic agent is xylazine HCl.

The therapeutically effective dose can be, for example, administered to the subject before onset of a retinal-degenerative disease. Additionally or alternatively, the therapeutically effective dose can be administered to the subject at or near onset of the retinal-degenerative disease, or after onset of the retinal-degenerative disease.

In certain embodiments, the therapeutically effective dose of the therapeutic agent can comprise at least two therapeutically effective doses administered to the subject over a period of at least two days. Alternatively, the therapeutically effective dose of the therapeutic agent can comprise at least two doses administered to the subject over a period of time (e.g., minutes, hours, or days) that, when taken together, equate to a therapeutically effective dose (e.g., dose loading).

The therapeutically effective dose can be administered to an eye of the subject (e.g., intraocularly). In certain embodiments, the therapeutically effective dose is administered in a manner that facilitates delivery of the therapeutically effective dose to a retina of the eye of the subject. In certain embodiments, the therapeutically effective dose is administered topically. In certain embodiments, the therapeutically effective dose is formulated as topical eye drops. In certain embodiments, the therapeutically effective dose is administered to the subject during early retinal development.

In certain embodiments, the method is for rod genesis (e.g., neogenesis) and the therapeutically effective dose is administered to the subject during early retinal development. In certain embodiments, the method is for rod regeneration and the therapeutically effective dose is administered to a mature subject.

The subject can be experiencing, or at risk for experiencing, a retinal-degenerative disease (e.g., retinitis pigmentosa (RP)). In certain embodiments, the therapeutically effective dose is administered at or near onset of a retinal-degenerative disease. In certain embodiments, the therapeutically effective dose is administered before onset of a retinal-degenerative disease (e.g., prophylactically).

The method can further comprise administering at least one additional therapeutic, pharmaceutical, biochemical, or biological agent or compound to the subject (e.g., for treatment of a retinal-degenerative disease or other related disorder). In certain embodiments, the at least one additional therapeutic, pharmaceutical, biochemical, or biological agent or compound is administered to the eye of the subject.

Methods for treating RP are also provided. In certain embodiments, a method for treating RP comprises administering a therapeutically effective dose of esmolol hydrochloric acid, triamterene, trelagliptin, prednisolone acetate, crenolanib, dolutegravir, tivantinib, noradrenaline bitartrate monohydrate, vidofludimus, gabapentin, gemcitabine HCl, desvenlafaxine succinate, LCZ696 or sacubitril/valsartan, Palbociclib (PD0332991) isethionate, galanthamine HBr, amitriptyline HCl, or xylazine HCl to an afflicted eye of the subject. The therapeutically effective dose can be administered topically, intraocularly, or systemically. The therapeutically effective dose can be administered to the subject before, at or near, or after onset of RP. Such methods can further comprise administering at least one additional therapeutic, pharmaceutical, biochemical, or biological agent or compound to the afflicted eye of the subject.

In the methods for treating RP, the therapeutically effective dose can comprise at least two therapeutically effective doses administered to the subject over a period of at least two days. The therapeutically effective dose can be formulated as topical eye drops. In certain embodiments, the therapeutically effective dose is administered to the subject during early retinal development.

Methods for the in vitro production of a population of rod photoreceptors are also provided. Such methods can comprise culturing retinal progenitor cells under conditions and for a period of time that enable cell growth and differentiation of the retinal progenitor cells to produce photoreceptor progenitor cells (e.g., rod progenitor cells), wherein the conditions comprise exposure to a therapeutically effective dose of a therapeutic agent selected from the group consisting of carvedilol, esmolol hydrochloric acid, triamterene, trelagliptin, prednisolone acetate, crenolanib, dolutegravir, tivantinib, noradrenaline bitartrate monohydrate, vidofludimus, gabapentin, gemcitabine HCl, desvenlafaxine succinate, LCZ696 or sacubitril/valsartan, Palbociclib (PD0332991) isethionate, galanthamine HBr, amitriptyline HCl, and xylazine HCl.

The method can further comprise culturing pluripotent stem cells to produce one or more retinal progenitor cells. Additionally or alternatively, the method can further comprise dissociating an extracellular matrix of retinal tissue or retinal tissue fragments from a subject so to dissociate retinal progenitor cells from each other without lysing the retinal progenitor cells. There, the retinal tissue or retinal tissue fragments can be mammalian retinal tissue or mammalian retinal tissue fragments, for example.

Populations of photoreceptor progenitor cells or photoreceptor rods generated therefrom are also provided, where the photoreceptor progenitor cells are obtained by the methods provided herein.

Pharmaceutical products are also provided, such pharmaceutical products comprising a population of progenitor cells or photoreceptor rods described herein. Uses of the pharmaceutical products are also provided. In certain embodiments, a use of the pharmaceutical products described herein comprises use in the manufacture of a medicament for the treatment of a retinal-degenerative disease. In certain embodiments, the retinal-degenerative disease is RP.

### BRIEF DESCRIPTION OF DRAWINGS

FIG. 1A shows a schematic of a visual motor response (VMR) protocol where, on 7 days post-fertilization (dpf), larvae were habituated to the machine in darkness for 30 minutes, followed by light stimulation with the plate illuminated for 60 minutes. Thereafter, the light was turned off. VMR was mainly analyzed at light offset (light-off VMR) as indicated by the arrow.

FIG. 1B is a graph of the light-off VMR of wildtype (WT, black trace) and Q344X (red trace) larvae at 0.01x. The light was turned off at Time=0. Each trace shows the average larval displacement of 18 biological replicates with 48 larvae per condition per replicate. The corresponding color ribbon indicates ±1 standard error of the mean (s.e.m.).

FIG. 1C shows a boxplot of the average larval displacement of WT and Q344X larvae one second after light offset. The average displacement of WT larvae (μ±s.e.m.: 0.281±0.036 cm, N=18) (Welch's Two Sample t-test, T=13.2, df=33.2, p value <0.0001). To confirm this scotopic VMR was driven by rods, rods in larvae were chemically-ablated and subjected to the same scotopic VMR assay (see FIGS. 1D and 1E).

FIG. 1D shows the data of light-off VMR of larvae with nitroreductase-expressing rods treated with metronidazole (MTZ) (rho:NTR+MTZ, red trace) and without MTZ (rho:NTR, black trace). Each trace shows the average displacement of 6 biological replicates with 24 larvae per condition per replicate. The corresponding color ribbon indicates ±1 s.e.m.

FIG. 1E shows a boxplot of the average displacement of rho:NTR and rho:NTR+MTZ larvae one second after light offset, with the average displacement of untreated rho:NTR larvae (μ±s.e.m): 0.317±0.061 cm, N=6) was significantly larger than that of rho:NTR+MTZ larvae (0.110±0.062 cm, N=6) (Welch's Two Sample t-test, T=5.9, df=10, p value <0.0001).

FIG. 2A shows a graph of data relating to carvedilol treatment on Q344X larvae (blue trace, N=2 replicates of 24 larvae) compared to that of both DMSO-treated WT larvae and DMSO-treated Q344X larvae (black and red trace respectively, N=9 replicates of 48 larvae in each group). Each trace shows the average displacement of each replicate, and the color ribbons indicate μ±s.e.m. The two carvedilol replicates were highly consistent and not different from each other (High-Dimensional Nonparametric Multivariate Test, N=24, T_{HD}=1.78, p value=0-91). Each replicate demonstrated a significant change in behavior for the duration of 30 seconds after light offset above DMSO-treated Q344X larvae (Hotelling's T-squared test. N=24, T=378.0 and 456.0, df=30, p value <0.0001 for each replicate).

FIG. 2B shows graphical data from eyeless chokh fish treated with carvedilol (blue trace) and untreated control (black trace) to determine if carvedilol's effects are elicited through the retina (Hotelling's T-squared test, N=24, T=37.8, df=30, p value=0.946).

FIG. 2C shows graphical data related to Q344X larvae being enucleated to determine if extraocular expression of Q344X RHO was causing the VMR seen with carvedilol treatment, with larvae treated with carvedilol (blue trace) or DMSO (red trace) at 5 dpf and enucleated on the morning of 6 dpf. VMR was assessed at 7 dpf (Hotelling's T-squared test, N=24, T=28.8, df=30, p value=0.948).

FIG. 2D shows graphical data from Q344X larvae treated with 100 μM adenylyl cyclase (ADCY) inhibitor SQ 22.536 (black trace) at 3 dpf to determine if inhibiting ADCY would improve the VMR as compared to DMSO treatment (red trace) (Hotelling's T-squared test, N=3. T=118, df=30, p value <0.0001).

FIG. 3A shows an image of wildtype larva (WT), FIG. 3B shows an image of a DMSO-treated Q344X larva, and FIG. 3C shows an image of a carvedilol-treated Q344X (car) larva at 7 dpf. Rods were labeled by EGFP expression driven by rho promoter, and the nuclei were counterstained with DAPI (scale=50 μm).

FIG. 3D shows quantification of rod number in WT, DMSO-treated Q344X, and carvedilol-treated Q344X retinal cryosections from 5 to 7 dpf. A statistically significant difference in rod number between groups at all stages was determined by one-way ANOVA at 5 dpf (WT, N=11; Q344X, N=16; F(1,25)=71.04, p value <0.0001), at 6 dpf (WT, N=9; Q344X, N=20; Q344X+car, N=11; F(2,44)=96.9, p value <0.0001), and at 7 dpf (WT, N=9; Q344X, N=17; Q344X+car, N=11; F(2,41)=167.9, p value <0.0001).

FIG. 3E are representative whole-eye images of WT (representative of the “Strong” group in Table 2), Q344X (representative of the “Weak” group in Table 2), and carvedilol-treated Q344X larvae at 7 dpf (Q344X+car) (representative of the “Intermediate” group in Table 2). Rods were labeled by EGFP expression. Left column: WT rods were mainly found on dorsal and ventral retina (top) and were abundantly present in the ventral patch of the retina extending medially (bottom). Middle column: Q344X rods were mostly degenerated at the same stage (top), with only a handful of rods remaining near the lateral edge of the ventral patch in the Q344X retina (bottom). Right column: carvedilol treatment increased the number of Q344X rods on both dorsal and ventral retina (top), with gaps of missing rods still apparent on dorsal retina and more rods observed in the ventral patch of the carvedilol-treated retina (bottom). Statistical analysis of whole-mount data is shown in Table 2 (scale=100 μm. D dorsal, V ventral, M medial, L lateral).

FIG. 4A shows the quantification of rod number in WT, Q344X treated with DMSO beginning at 3 dpf, and Q344X treated with carvedilol beginning at 3 dpf or 5 dpf Rods were quantified from their retinal cryosections beginning at 3-7 dpf. There was no statistically significant difference in rod number between groups at 3 dpf and 4 dpf determined by one-way ANOVA (3 dpf; N=10; F(3,36)=0.1, p value=0.95); (4 dpf; N=10; F(3,36)=0.1, p value=0.69). There was a statistically significant difference in rod number between groups at 5 dpf through 7 dpf determined by one-way ANOVA (5 dpf; N=10; F(3,36)=0.1, p value <0.0001); (6 dpf; N=10; F(3,36)=0.1, p value <0.0001); (7 dpf; N=10; F(3,36)=0.1, p value <0.0001). The effect of Q344X rod degeneration and carvedilol treatment on rod number was assessed post hoc by pairwise t-test with false discovery rate correction at 5 dpf (WT-Q344X, p value <0.0001; Q344X-Q344X+car3dpf, p value <0.001; Q344X-Q344X+car5dpf, p value=0.36, Q344X+car3dpf-Q344X+car5dpf, p value <0.0001), at 6 dpf (WT-Q344X, p value <0.0001; Q344X-Q344X+car3dpf, p value <0.0001; Q344X-Q344X+car5dpf, p value <0.05; Q344X+car3dpf-Q344X+car5dpf, p value <0.05), and at 7 dpf (WT-Q344X, p value <0.0001; Q344X-Q344X+car3dpf, p value <0.0001; Q344X-Q344X+car5dpf, p value <0.05; Q344X+car3dpf-Q344X+car5dpf, p value <0.05).

FIG. 4B shows supports carvedilol treatment of Q344X larvae beginning at 3 dpf (purple trace) displayed a significant scotopic light-off VMR when compared to Q344X larvae treated with DMSO (red trace) (Hotelling's T-squared test. N=3 replicates of 24 larvae. T=397, df=30, p value <0.0001). Each trace shows the average displacement of each replicate and the color ribbons indicate μ±s.e.m.

FIG. 5A shows representative dose-response curves of GloSensor-transfected Y79 cells treated with half-log concentrations of isoproterenol (red trace; N=4) or percentage-matched DMSO (black trace; N=4) (plots normalized to the maximum average luminescent level recorded per experiment; errors bars shown ±s.e.m. Isoproterenol was capable of increasing cAMP signaling through β-adrenergic receptor binding with an pEC50 of 7.49±1.07.

FIG. 5B shows representative dose-response curves of GloSensor-transfected Y79 cells pretreated with half-log doses of carvedilol (blue trace; N=4) or percentage-matched DMSO (red trace; N=4). Cells were then challenged with a 10 μM isoproterenol to induce maximal cAMP response, as shown in FIG. 5A.

FIG. 6 shows graphical data from a rod development assay that measured rod development in a control group (DMSO), a group treated with 10 μM carvedilol (CAR) at 3-7 days post fertilization (dpf) (labeled CAR), and a positive control group treated with 1.2 μM of retinoic acid at 3-7 dpf.

FIG. 7 shows boxplot data from the rod neogenesis assay with drug treatment administered 5-7 dpf assay (n=96 in each group) (YFP intensity ratios (μ±SD): CAR/DMSO=1.8±0.9, RA/DMSO=1.61±1.35; Student's t-test, Bonferroni-corrected p=1.82e-10 and 9.25e-5, respectively).

FIG. 8 shows boxplot data from a rod regeneration assay that measured rod regeneration following a 10 mM metronidazole (MTZ) challenge at day 5 dpf. −0.1% DMSO=control group, no ablation/treatment with MTZ, treatment with 0.1% DMSO; +0.1% DMSO=control group, treatment with MTZ and treatment with 0.1% DMSO; +2.5 μM prednisolone (PRE)=positive control group; +2.5 μM dexamethasone (DEX)=positive control group; +10 μM CAR=treatment group. (CAR YFP intensity ratio: CAR/DMSO=1.12±0.81; Student's t-test, Bonferroni corrected p=0.41; DEX YFP intensity ratio: DEX/DMSO=0.65-0.48; Student's t-test, Bonferroni corrected p=0.001). All ablated groups also had fewer rods than the unablated controls.

FIG. 9 shows graphical data from the rod development assay of FIG. 8, with drug treatment administered 6-9 dpf.

FIG. 10A shows boxplots of larval VMR with and without drug treatment, with the 8 boxes on the left representative of the 8 drug hits identified (n=2 replicates of 24 larvae for each hit), showing that the 8 drug hits significantly increased the first second of the light-Off VMR of the Q344X larvae as compared with the control Q344X larvae exposed to DMSO (second box from the right labeled Q344X+DMSO; n=9 replicates of 48 larvae) (Welch's Two Sample t-test, Bonferroni adjusted p-value <0.05). The increased activity of these hits is also comparable to that in the WT (far right box; n=9) replicates of 48 larvae).

FIG. 10B represents the VMR trace of one of the hits (p9g2 or noradrenaline bitartrate monohydrate) (C) compared with that of the WT (A) and Q344X (B) controls exposed to DMSO at 7 dpf (note the activity peak at the first second overlaps between WT and Q344X exposed to the p9g2 (noradrenaline bitartrate monohydrate) compound hit.

FIG. 11 shows graphical data related to an optimized light-On VMR for detecting rod response in the Q344X mutant, with dark adaptation time extended before the light onset. A consistent difference was detected between the WT and Q344X mutant during the light-On VMR for rod response at 7 dpf (traces representing average displacement of larvae from 6 biological replicates with 48 larvae per genotype per replicate).

FIGS. 12A and 12B show data related to new drug hits for Q344X adRP identified by screening an FDA library. FIG. 12A shows hierarchical clustering (HC) of the drug hits of FIG. 10A that induced the Q344X mutant to display a WT-like VMR. FIG. 12B shows a VMR profile induced by amitriptyline (p10h6) that was highly similar to the WT profiles (Hotelling's T² test, p=0.41).

FIG. 13 shows data related to CAR and the promotion of rod neogenesis. FIG. 13 is a bloxplot of cell death as indicated by a TUNEL assay on cryosections.

FIG. 14 shows images of the ventral view of a representative eye for each treatment group of a study where nr2e3 expression was detected by in situ hybridization (ISH) (n=10), with lateral (L) to the left and medial (M) to the right. The CMZ is located next to the lens. In WT, nr2e3-expressing cells (dark grey) were detected in the medial edge of the CMZ, and in a ventral patch (VP), a region of precious neural development where a substantial number of photoreceptors can be found. Fadool & Dowling. Zebrafish: a model system for the study of eye genetics. Prog Retinal Eye Research 27: 89-110 (2008). This VP was not obvious in the Q344X and the Q344X+CAR groups; nonetheless, more nr2e3-expressing cells were detected in the medial edge of the CMZ in the Q344X+CAR group as compared to the Q344X group, suggesting more rods were specified.

It is to be understood that the drawings are not intended to limit the scope of the present teachings in any way.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application. As previously noted, while this technology may be illustrated and described in one or more preferred embodiments, the compositions, compounds, and methods hereof may comprise many different configurations, forms, materials, and accessories.

Methods for Photoreceptor Rod Regeneration and/or Rod Genesis

Methods for photoreceptor rod regeneration or rod genesis are provided. Such methods can facilitate the enhanced regeneration or genesis of photoreceptors as compared to regeneration or genesis rates observed in a comparable control subject that did not receive the treatment. Rather than holding back existing photoreceptors' degeneration to, for example, curtail or cease the progression of a retinal-degenerative disease state, this therapy can facilitate the genesis of new photoreceptors (e.g., in subjects with resident stem cells present such as, for example, in a fetus) or the regeneration of existing photoreceptors (e.g., in mature subjects). As used herein, the term “mature” means a subject that does not have a significant amount of progenitor cells naturally present in the eye.

The method can comprise administering, to a subject, a therapeutically effective dose of a therapeutic agent. The therapeutic agent can comprise a β-blocker. In certain embodiments, the β-blocker is carvedilol. In certain embodiments, the β-blocker is metipranolol, metoprolol, nebivolol, or an atypical β-blocker. In certain embodiments, the therapeutic agent is selected from the group consisting of carvedilol, esmolol hydrochloric acid (p11b7), triamterene (p11c5), trelagliptin (p11c7), prednisolone acetate (p8b10), crenolanib (p9a4), dolutegravir (p9c3), tivantinib (p9c4), noradrenaline bitartrate monohydrate (p9g2), vidofludimus (p15f5), gabapentin (p16g10), gemcitabine hydrochloride (HCl) (p16d6), desvenlafaxine succinate (p11b8), LCZ696 or sacubitril/valsartan (p15g8), Palbociclib (PD0332991) isethionate (p16c8), galanthamine hydrobromide (HBr) (p16h6), amitriptyline HCl (p10h6), and xylazine HCl (p8g5). In certain embodiments, the method comprises administering, to a subject, a therapeutically effective dose of a therapeutic agent comprising p9g2. A “subject” can be a human patient, a laboratory animal, such as a rodent (e.g., mouse, rat, or hamster), a rabbit, a monkey, a chimpanzee, a fish, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, a whale or a fish.

As noted above, the therapeutically effective dose of the therapeutic agent can be administered to the subject (e.g., administered to an afflicted eye for delivery to a retina of the subject) to facilitate genesis of new photoreceptor rods (e.g., in a retina of the subject). For example, and without limitation, the therapeutically effective dose of the therapeutic agent (e.g., carvedilol) can be administered to a subject in need of additional photoreceptor rods (such as where the subject is actively experiencing a retinal-degenerative disease (e.g., a disease that affects the growth of photoreceptor rods and/or results in photoreceptor rod degeneration in an eye)). In certain instances, the therapeutically effective dose of the therapeutic agent can be administered to a subject prophylactically to facilitate enhanced photoreceptor genesis (e.g., where a subject is at risk of experiencing a retinal-degenerative disease).

The subject can have (or be at risk of having) retinitis pigmentosa (RP), which can be associated with rod-cone retinal degenerations within the afflicted eye. The subject can have (or be at risk of having) autosomal dominant cases of RP (adRP).

### Methods for Treating RP

Methods for treating RP (e.g., adRP) are also provided. A method for treating RP can comprise administering a therapeutically effective dose of esmolol hydrochloric acid (p11b7), triamterene (p11c5), trelagliptin (p11c7), prednisolone acetate (p8b10), crenolanib (p9a4), dolutegravir (p9c3), tivantinib (p9c4), noradrenaline bitartrate monohydrate (p9g2), vidofludimus (p15f5), gabapentin (p16g10), gemcitabine hydrochloride (HCl) (p16d6), desvenlafaxine succinate (p11b8), LCZ696 or sacubitril/valsartan (p15g8), Palbociclib (PD0332991) isethionate (p16c8), galanthamine hydrobromide (HBr) (p16h6), amitriptyline HCl (p10h6), or xylazine HCl (p8g5) to an afflicted eye of the subject.

In each of the methods, a therapeutically effective dose of the therapeutic agent can be administered to the subject prophylactically (e.g., before disease onset) or therapeutically (e.g., concurrently or after disease onset). In certain embodiments, the therapeutically effective dose is administered to the subject at least 3 days post-fertilization (dpf) of the subject, 5 dpf of the subject, and/or 7 dpf of the subject. The therapeutically effective dose can also, for example, comprise at least two therapeutically effective doses administered to the subject over a period of several hours or days (e.g., over a period of at least two days).

The carvedilol and other therapeutic agents described herein can be formulated for therapeutic or research use. Typically, such formulations for therapy include the therapeutic agent (e.g., carvedilol) suspended in a pharmaceutically acceptable carrier.

The therapeutic agents can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the subject including, but not limited to, intraocular, systemic, and like routes of administration. In certain embodiments, the therapeutic agent or composition is formulated into topical eye drops (e.g., for application to a retina of a subject) and applied locally to the eye of the subject (e.g., intraocularly).

As used herein, the term “composition” generally refers to any product comprising more than one ingredient, including the therapeutic agent (e.g., carvedilol).

The therapeutic agent can be formulated as pharmaceutical composition (e.g., a pharmaceutical product) and administered to a mammalian host, such as a human patient, in a variety of forms adapted to the chosen route of administration. For example, the pharmaceutical composition can be formulated for and administered via oral, topical, or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. In at least one embodiment, carvedilol, such as part of a composition, can be administered directly into the eye or onto retina. In certain embodiments, the carvedilol, such as part of a composition, can be administered directly to an embryo or a larva.

For example, in at least one embodiment, a therapeutic agent is systemically administered in combination with a pharmaceutically acceptable vehicle. The vehicle can be a pharmaceutically acceptable carrier. The phrases “pharmaceutically acceptable carrier” and “carrier” are used interchangeable and mean one or more compatible solid or liquid fillers, diluents or encapsulating substances which are suitable for administration to a human or other animal. The carrier can be an excipient.

A pharmaceutically acceptable carrier can include any and all solvents, dispersion media, coatings, antibacterial and antifungal agents, isotonic and absorption delaying agents, and the like, and combinations thereof, that are physiologically compatible. The carrier can be suitable for parenteral administration. Pharmaceutically acceptable carriers include sterile aqueous solutions or dispersions and sterile powders for the extemporaneous preparation of sterile injectable solutions or dispersions. Examples of such carriers (or excipients) include, but are not limited to, calcium carbonate, calcium phosphate, various sugars, starches, cellulose derivatives, gelatin, and polymers such as polyethylene glycols. One or more other active agents also can be incorporated into a pharmaceutical composition.

Excipients can include suspending agents, such as sodium carboxymethylcellulose, methylcellulose, hydroxypropyl methylcellulose, sodium alginate, polyvinylpyrrolidone, gum tragacanth and gum acacia; dispersing or wetting agents, such as a naturally-occurring phosphatide, e.g., lecithin; a condensation product of an alkylene oxide with a fatty acid, e.g., polyoxyethylene stearate; a condensation product of ethylene oxide with a long-chain aliphatic alcohol, e.g., heptadecaethyleneoxcycetanol; a condensation product of ethylene oxide with a partial ester derived from fatty acids and a hexitol, such as polyoxyethylene sorbitol monooleate; or a condensation product of ethylene oxide with a partial ester derived from fatty acids and hexitol anhydrides, e.g., polyoxyethylene sorbitan monooleate. The aqueous suspension also can contain one or more preservatives, e.g., ascorbic acid or ethyl, n-propyl, or p-hydroxy benzoate, and one or more coloring agents. In certain embodiments, an aqueous suspension can further comprise suitable lipophilic solvents or vehicles including fatty oils such as sesame oil, or synthetic fatty acid esters, such as ethyl oleate or triglycerides, or liposomes.

The percentages of the components of the compositions and preparations can vary and can be between about 1 to about 99% weight of the active ingredient(s) and a binder, excipients, a disintegrating agent, a lubricant, and/or a sweetening agent (as are known in the art). The amount of active compound (e.g., therapeutic agents) in such therapeutically useful compositions is such that an effective dosage level can be obtained.

The preparation of parenteral compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the compositions can also be administered topically or via infusion or injection (e.g., using needle (including microneedle) injectors and/or needle-free injectors). Solutions of the active composition can be aqueous, optionally mixed with a nontoxic surfactant and/or contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations may further contain a preservative to prevent the growth of microorganisms.

In certain embodiments, the compositions comprise aqueous solutions such as, for example, physiological saline, oil, gels, patches, solutions, or ointments. The vehicles that carry these biologically active therapeutic agents can contain conjunctivally compatible preservatives (such as, for example, benzalkonium chloride) and/or surfactants such as, for example, polysorbate 80, liposomes or polymers (such as, for example, methyl cellulose, polyvinyl alcohol, polyvinyl pyrrolidone, hyaluronic acid and the like).

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants.

Sterile injectable solutions can be prepared by incorporating the therapeutic agents (e.g., carvedilol or p9g2) and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

For topical administration, it can be desirable to administer the compositions and/or therapeutic agent (e.g., carvedilol or p9g2) directly to an eye (e.g., for delivery to a retina) as compositions or formulations in combination with an acceptable carrier, which may be a solid or a liquid. For example, in certain embodiments, solid carriers may include finely divided solids such as saline, talc, clay, microcrystalline cellulose, silica, alumina and the like. Similarly, useful liquid carriers may comprise water or glycols or water-alcohol/glycol blends, in which the present compounds can be dissolved or dispersed at effective levels, optionally with the aid of non-toxic surfactants. Additionally or alternatively, adjuvants such as antimicrobial agents can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the terms “therapeutically effective.” “therapeutically effective dose,” or “therapeutically effective amount” (unless specifically stated otherwise) a quantity of a therapeutic agent (e.g., carvedilol, esmolol hydrochloric acid (p11b7), triamterene (p11c5), trelagliptin (p11c7), prednisolone acetate (p8b10), crenolanib (p9a4), dolutegravir (p9c3), tivantinib (p9c4), noradrenaline bitartrate monohydrate (p9g2), vidofludimus (p15f5), gabapentin (p16g10), gemcitabine hydrochloride (HCl) (p16d6), desvenlafaxine succinate (p11b8), LCZ696 or sacubitril/valsartan (p15g8), Palbociclib (PD0332991) isethionate (p16c8), galanthamine hydrobromide (HBr) (p16h6), amitriptyline HCl (p10h6), or xylazine HCl (p8g5)) and/or a compound (e.g., a therapeutic agent) that, when administered either one time or over the course of a treatment cycle, affects or otherwise promotes the genesis of photoreceptor rods of a subject (e.g., and without limitation, delays the onset of and/or reduces the severity of one or more of the symptoms associated with a retinal-degenerative disease such as RP). In certain embodiments, a therapeutically effective amount can provide a prophylactic effect (e.g., when administered before or near onset of a retinal-degenerative diseases).

Useful dosages of the therapeutic agents can be determined by comparing their in vitro activity with their in vivo activity in animal models. Methods of the extrapolation of effective dosages in mice and other animals to human subjects are known in the art. Indeed, the dosage of the therapeutic agent can vary significantly depending on the condition of the host subject, the age of the subject, the type retinal-degenerative disease the subject is experiencing or at risk of experiencing, the particular β-blocker used, how advanced the pathology is, the route of administration of the compound and tissue distribution, and the possibility of co-usage of other therapeutic treatments (such as cell-based therapy (e.g., stem-cell infusion therapy) or additional drugs in combination therapies). The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

Therapeutically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total therapeutically effective amount of the therapeutic agent can be administered in single or divided doses and can, at the practitioner's discretion, fall outside of the typical range given herein.

In certain embodiments, a therapeutically effective dose of a drug can be a dose of the therapeutic agent (e.g., for humans) approved by the U.S. Federal Drug Administration (FDA). For example, carvedilol is an FDA-approved drug, which means the FDA has deemed it safe for use in humans. Carvedilol does not appear to cause eye issues when administered repeatedly to rabbits, can lower intraocular pressure (IOP), and does not appear to have an effect on iris or retina/choroid blood flow.

In another embodiment, carvedilol can be administered in a therapeutically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m².

### Methods for In Vitro Production of a Population of Rod Photoreceptors, Products, and Uses Thereof

A method for the in vitro production of a population of rod photoreceptors is also provided. In certain embodiments, such a method comprises culturing retinal progenitor cells under conditions and for a period of time that enable cell growth and differentiation of the cells to produce photoreceptor progenitor cells, wherein the conditions include exposure to a therapeutically effective dose of a therapeutic agent described herein (e.g., a β-blocker, a carvedilol, esmolol hydrochloric acid (p11b7), triamterene (p11c5), trelagliptin (p11c7), prednisolone acetate (p8b10), crenolanib (p9a4), dolutegravir (p9c3), tivantinib (p9c4), noradrenaline bitartrate monohydrate (p9g2), vidofludimus (p15f5), gabapentin (p16g10), gemcitabine hydrochloride (HCl) (p16d6), desvenlafaxine succinate (p11b8), LCZ696 or sacubitril/valsartan (p15g8), Palbociclib (PD0332991) isethionate (p16c8), galanthamine hydrobromide (HBr) (p16h6), amitriptyline HCl (p10h6), or xylazine HCl (p8g5)). The therapeutically effective dose of the therapeutic agent can be a dose sufficient to facilitate enhanced genesis of photoreceptor rods from the photoreceptor progenitor cells as compared to in vitro cultured photoreceptor progenitor cells that are not exposed to the therapeutically effective dose of the therapeutic agent. In certain embodiments, the method further comprises culturing pluripotent stem cells to produce one or more retinal progenitor cells (e.g., rod progenitor cells).

Photoreceptor development can occur through a number of developmental stages, each of which can be defined phenotypically (e.g., by way of a marker expression profile) and/or functionally. In vitro pluripotent stem cells can differentiate into eye field progenitors, which in turn can differentiate into photoreceptor progenitor cells, which in turn can differentiate into photoreceptor cells and/or generate photoreceptor rods. “Progenitor cells” as used herein means cells that can produce more progenitor cells of the same or of more limited differentiative capacity or can differentiate to an end fate cell lineage (e.g., photoreceptor rods).

The method can further comprise dissociating an extracellular matrix of retinal tissue or retinal tissue fragments from a subject so to dissociate retinal progenitor cells from each other without lysing the retinal progenitor cells. The retinal tissue or retinal tissue fragments can be mammalian retinal tissue or mammalian retinal tissue fragments.

Populations of photoreceptor progenitor cells obtained by the aforementioned methods and/or photoreceptor rods regenerated or neogenerated therefrom are also provided. For example, the photoreceptor progenitor cells and/or photoreceptor rods can be used in vivo to treat conditions of the retina, including but not limited to RP. In certain embodiments, the subject is mature, and the method comprises a method for photoreceptor rod regeneration. In certain embodiments, the subject is a fetus or otherwise comprises progenitor cells in its eye and the method comprises a method for photoreceptor rod genesis (i.e., neogenesis means the generation of new rods). The photoreceptor progenitor cells can be used in vitro in screening assays to identify putative therapeutics or prophylactic treatment candidates.

Further provided is a pharmaceutical composition comprising the photoreceptor progenitor cells, derivatives thereof, and/or photoreceptor rods obtained using the methods described herein. Such pharmaceutical composition can further comprise a vehicle as described herein in connection with pharmaceutical compositions generally.

The choice of carrier can depend on factors such as the particular mode of administration, the effect of the carrier on solubility and stability, and the nature of the dosage form. Pharmaceutical compositions suitable for the delivery of compounds as described herein and methods for their preparation may be found, for example, in Remington: The Science & Practice of Pharmacy. 21st edition (Lippincott Williams & Wilkins, 2005).

The concentration of photoreceptor progenitor cells, derivatives thereof, and/or photoreceptor rods in the pharmaceutical composition can be about 200 cells or more per microliter. In certain embodiments, the concentration of photoreceptor progenitor cells, derivatives thereof, and/or photoreceptor rods in the pharmaceutical composition is between about 2.000 and about 5,000 cells per microliter (such as 1,000-5,000 cells/microliter, about 2,000-5,000 cells/microliter, or 2,000-about 5,000 cells/microliter).

Pharmaceutical compositions can be prepared by combining one or more photoreceptor progenitor cells, derivatives thereof, and/or photoreceptor rods with a pharmaceutically acceptable carrier and, optionally, one or more additional ingredients (e.g., pharmaceutically active ingredients). The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

The pharmaceutical composition can be formulated as a liquid, e.g., a suspension or a solution. A liquid formulation can comprise water, ethanol, PEG, propylene glycol, methylcellulose, or a suitable oil, and one or more emulsifying agents and/or suspending agents. A liquid formulation can be prepared by the reconstitution of a solid. In certain embodiments, the composition is suitable for (i.e., comprises a formulation suitable for) intraocular injection.

Pharmaceutical formulations can include suspensions of the photoreceptor progenitor cells, derivatives thereof, and/or photoreceptor rods which are prepared as appropriate oily or water-soluble injection suspensions. An aqueous suspension can contain the photoreceptor progenitor cells, derivatives thereof, and/or photoreceptor rods, alone or in further combination with one or more other active agents, in an admixture with an appropriate excipient.

The components of the compositions also can be commingled with the photoreceptor progenitor cells, derivatives thereof, and/or photoreceptor rods, and with each other, in a manner such that there is no interaction which would substantially impair the desired physiological efficiency.

The composition can comprise cremophor, polysorbate, nanoparticles, a polymer, or a hydrogel, for example. In certain embodiments, a pharmaceutical composition further comprises at least one additional pharmaceutically active agent.

Pharmaceutical compositions can be prepared by combining one or more photoreceptor progenitor cells, derivatives thereof, and/or photoreceptor rods with a pharmaceutically acceptable carrier and, optionally, one or more additional ingredients (e.g., pharmaceutically active ingredients). The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

In certain embodiments, a therapeutically effective dose of one or more therapeutic agents hereof (including, without limitation, a pharmaceutical composition hereof) can be administered intravitreally in the following doses: 0.1% (2.4 mmol/L), 0.5% (12.3 mmol/L), 1% (24 mmol/L), respectively (N=3-5). See Szumny and Szelag, The influence of new beta-adrenolytics nebivolol and carvedilol on intraocular pressure and iris blood flow in rabbits, Gruefes Arch Clin Exp Ophthalmol 252:917-23 (2014).

These and other effective unit dosage amounts can be administered in a single dose, or in the form of multiple hourly, daily, weekly, or monthly doses, for example in a dosing regimen of once per day for a 3-day cycle. In additional embodiments, dosages can be administered in concert with other treatment regimens in any appropriate dosage regimen depending on clinical and patient-specific factors. The amount, timing, sequence, and mode of delivery of compositions comprising a disease-treating effective amount (e.g., a therapeutically effective amount) of a therapeutic agent will be routinely adjusted on an individual basis, depending on such factors as weight, age, gender, and condition of the individual, the acuteness of the disease and/or related symptoms, whether the administration is prophylactic or therapeutic, and on the basis of other factors known to effect drug delivery, absorption, pharmacokinetics including half-life, and efficacy.

Uses of the pharmaceutical compositions hereof in the manufacture of a medicament for the treatment of a retinal-degenerative disease are also provided. In at least one embodiment, the pharmaceutical composition comprises photoreceptor progenitor cells, derivatives thereof, and/or photoreceptor rods as obtained using the methods (e.g., the in vitro methods) hereof. In certain embodiments, the retinal-degenerative disease is retinitis pigmentosa.

### Combination Methods

The method for photoreceptor rod regeneration or rod genesis and/or the method for treating RP can each further comprise administering at least one additional therapeutic, pharmaceutical, biochemical, or biological agent or compound to the afflicted eye of the subject. In certain embodiments, the at least one additional therapy comprises stem-cell therapy. In certain embodiments, the at least one additional therapy comprises prosthesis. In certain embodiments, the at least one additional therapy comprises gene therapy. In certain embodiments, the at least one additional therapy comprises administration of a vitamin supplement (e.g., a vitamin A supplement). In certain embodiments, the at least one additional therapy comprises administering a therapeutically effective amount of lutein, zeaxanthin, or docosahexaenoic acid (DHA).

The additional therapy(ies) can have a synergistic combination with the therapeutically effective dose of the therapeutic agent such that the therapeutic efficacy is greater than an additive. In certain embodiments, the combination therapy reduces or avoids unwanted or adverse effects.

For example, and without limitation, an additional therapy that can be administered can be purified platelet growth factor, insulin like growth factor-I (IGF-I), and/or other therapeutic agents to promote angiogenesis, increase the blood supply to the afflicted area, and otherwise enhance the healing process. In certain embodiments, the at least one additional therapy can be administration of prednisolone (e.g., 2.5 μM) or dexamethasone (e.g., 2.5 μM) to the subject. The at least one additional therapy can further comprise administration (e.g., locally or systemically) of at least one pain reducing and/or anti-inflammatory therapeutic agent. In certain embodiments, the additional therapy comprises administration of antiseptics, antibiotics, anti-virals, anti-fungals, anti-inflammatoires, steroids, and/or vasodilators to the afflicted eye. Such vasoconstrictors, for example, can include phenylephrine, ephedrine sulfate, epinephrine, naphazoline, neosynephrine, vasoxyl, oxymetazoline, or any combinations thereof. Such anti-inflammatoires can include non-steroidal anti-inflammatory drugs (NSAIDS), which can alleviate pain and inflammation. NSAIDS can, for example, include celecoxib, meloxicam, nabumetone, piroxicam, napmxen, oxaprozin, rofecoxib, sulindac, ketoprofen, valdewxid, anti-tumor necrosis factors, 10 anti-cytokines, anti-inflammatory pain causing bradykinins or any combination thereof.

The combination therapies (e.g., wherein the method comprises administering at least one additional therapeutic, pharmaceutical, biochemical, or biological agent or compound to the subject in addition to a therapeutically effective dose of a therapeutic agent) can provide an improved overall therapy relative to administration of a therapeutically effective dose of a therapeutic agent alone. In certain embodiments, doses of existing ophthalmic treatments can be reduced or administered less frequently where the therapeutic agents hereof increase generation of photoreceptor rods in the afflicted eye.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

### EXAMPLES

The following examples serve to illustrate the present disclosure. The examples are not intended to limit the scope of the claimed invention.

### Materials

### Zebrafish Model

A transgenic zebrafish autosomal dominant retinitis pigmentosa (adRP) model was used to perform phenotypic drug screening studies. Adult and larval zebrafish were maintained and bred using standard procedure. See Westerfield, The Zebrafish Book. A Guide for the Laboratory Use of Zebrafish (Danio rerio), 5^{th} edition, Univ. Oregon Press (2007). Adult fish began spawning at 9:00 am and embryos were collected before 10:30 am. Larval zebrafish were reared until 7 days post-fertilization (dpf) in E3 medium in an incubator at 28° C. The fish incubator was kept on a 14 hour light and 10 hour dark cycle. E3 medium was changed daily, and healthy embryos were kept for experiments.

The transgenic zebrafish expressed a truncated human rhodopsin transgene (Q344X) with autosomal dominant (ad) mutations found in human RP patients. See Ganzen et al., Drug screening with zebrafish visual behavior identifies carvedilol as a potential treatment for an autosomal dominant form of retinitis pigmentosa. Scientific Reports. Natural Portfolio 11:11432 (2021) (available at ttps://doi.org/10.1038/s41598-021-89482-z). Up to 30% of RP cases are autosomal dominant, and of all autosomal dominant cases, approximately 30% arise due to over 150 mutations in RHO. These mutations include Q344X/Q344ter, a truncation mutation, which shortens RHO at the C-terminus by 5 amino acids. Patients with this mutation suffer an early onset, severe form of adRP. Q344X RHO loses a VXPX ciliary trafficking motif on the C-terminus leading to its mislocalization to the inner segment and apoptotic cell death. Despite the C-terminal truncation, Q344X RHO remains a catalytically active protein and is capable of G protein signaling.

The transgenic zebrafish model expressed a truncated human rhodopsin transgene (Q344X RHO) in rods under the zebrafish rho promoter. Q344X larvae were identified on 2 dpf through the expression of EGFP under the control of 1.1 kbp promoter of olfactory marker protein (omp) contained in the transgenic cassette. Their genotype was verified via PCR. This model exhibited significant rod degeneration in the animals as early as 5 dpf.

Previous work with the Q344X zebrafish has shown that adenylyl cyclase (ADCY) inhibition can lead to modest rod survival. However, it was also shown that the activation of mislocalized RHO is not necessary to induce cell death. These findings indicate that Q344X can cause rod degeneration through more than one mechanism.

The transgenic fish larvae display a deficit in visual motor response (VMR) under scotopic conditions, which was leveraged to screen drugs in connection with retinal-degenerative disease treatment. Zebrafish rod precursors begin to differentiate into rods as early as 36 hours-post fertilization (hpf) in the ventral region of the retina by expressing rho. The rod outer segments begin to form by 50 hpf and fully formed outer segments have been identified as early as 4 dpf. These rods begin to form synapses by 62 hpf. The earliest visually-evoked startle can be detected by 68 hpf. After that, several visual behaviors gradually appear from 3 to 5 dpf, including the optokinetic response and the VMR. The VMR is a startle response triggered by a sudden light onset or offset, which results in increased locomotive behavior. This behavior can be measured from multiple larvae simultaneously in a 96-well plate format. The VMR has been utilized to identify oculotoxic drugs, and drugs that can benefit retinal degeneration. Zebrafish have also been used to perform high-throughput drug screening based on fluorescent signals in the retina, but this approach does not provide direct functional insight. On the contrary, utilizing the VMR as a drug-screening platform identifies compounds that improve visual function.

It was confirmed that the adRP model exhibited a diminished scotopic VMR behavior by 7 dpf, which was driven by rods (as confirmed by specific rod ablation). The diminished VMR of the transgenic Q344X zebrafish model was initially leveraged to screen an ENZO SCREEN-WELL REDOX library since oxidative stress is postulated to play a role in RP progression. The screen uncovered carvedilol, a β-adrenergic receptor antagonist, enhanced the Q344X zebrafish VMR. Carvedilol is already approved by the FDA to treat heart failure and high blood pressure and, as such, can be repurposed for the treatment of RP, for example.

### Example 1

### VMR Assay and Background Studies

The VMR assay was used to screen drugs with the Q344X zebrafish model (described above) using a scotopic light stimulus. This fish model was selected for drug screening as its rods begin to degenerate at 5 dpf, and the rod degeneration becomes severe by 7 dpf. The rapid rod degeneration facilitates rapid evaluation of many compounds on many individual larvae.

To determine the visual consequences of rod degeneration in the Q344X zebrafish, their VMR were measured under scotopic light illumination. An appropriate scotopic intensity was identified by systematically attenuating light intensity with neutral density filters until the light was 0.01 lx. To conduct the VMR assay. Q344X transgenic larvae were identified and sorted at 2 dpf by nose fluorescence. These larvae were dark adapted overnight at 6 dpf in a 96-well plate, and their VMR assessed at 7 dpf.

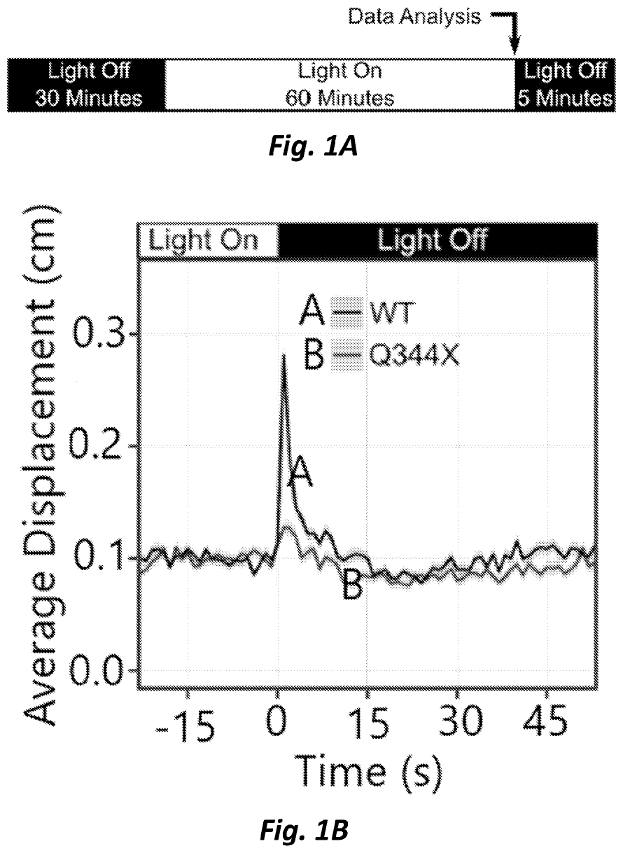

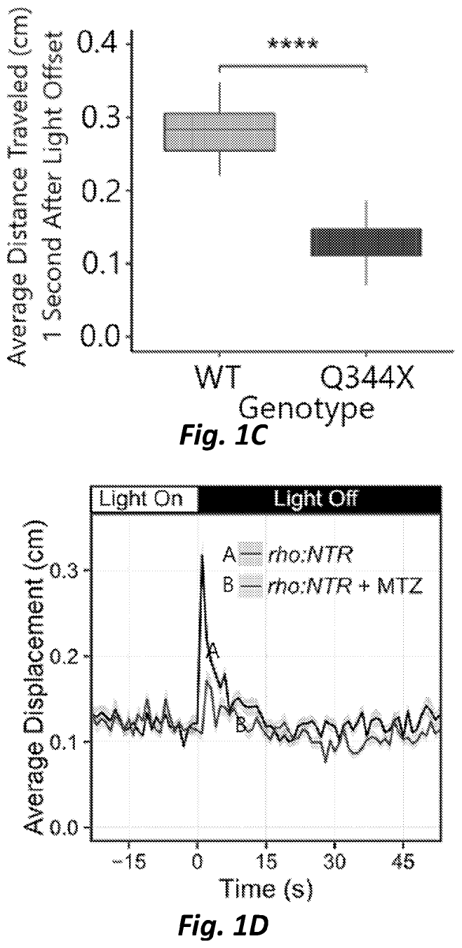

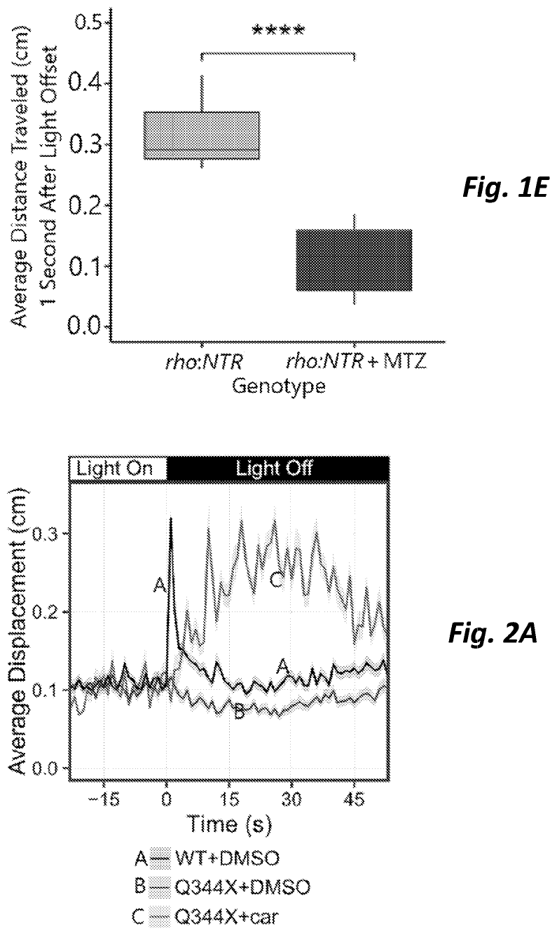

To conduct a VMR experiment, these larvae were acclimated to the machine in darkness for 30 minutes, exposed to the scotopic light of 0.01 lx for 60 minutes, and then exposed to darkness again (FIG. 1A). The larval displacement was recorded per second for the duration of the experiment. When exposed to a light intensity of 0.01 lx, wild-type (WT) larvae displayed a robust startle response immediately after light offset (light-off VMR), while Q344X larvae displayed a significantly diminished light-off VMR (FIG. 1B) Specifically, WT larvae traveled significantly further on average than the Q344X larvae (μ±standard error of the mean (s.e.m.): 0.281±0.036 cm vs. 0.127±0.031 cm) one second after light offset (FIG. 1C). Both Q344X and WT larvae did not show a response to the light onset at 0.01 lx, and both groups displayed a similar VMR at higher photopic intensities. These results support the expression of Q344X RHO diminished the light-off VMR of Q344X larvae at 0.01 lx. The VMR assay had a sensitivity of 83%, a specificity of 100%, and a positive predictive value of 100%. See Deeti et al., Early safety assessment of human oculotoxic drugs using the zebrafish visualmotor response, J Pharmacol Toxicol Methods 69:1-8 (2014).

It was thereafter confirmed rod degradation was responsible for the diminished scotopic VMR of Q344X larvae using rod ablation. To this end, a zebrafish line was used that expressed nitroreductase (NTR) specifically in rods under the control of the rhodopsin promotor (rho:NTR).

This enzyme converts a prodrug metronidazole (MTZ) into a cytotoxic substance and specifically ablates rods. In this study, the NTR-expressing larvae were treated with 2.5 mM MTZ (rho:NTR+MTZ) from 5 to 7 dpf, and their scotopic light-off VMR was measured at 7 dpf. Like the Q344X line, the rod-ablated larvae showed a significantly diminished light-off VMR compared with the untreated larvae (FIG. 1D). The average displacement of rho:NTR group (0.317±0.061 cm) was significantly further than that of rho:NTR+MTZ group (0.110±0.062 cm) (FIG. 1E). The reduction of scotopic light-off VMR by rod ablation indicates that the response was substantially driven by rods. The rho:NTR line displayed a strong VMR to photopic stimuli with and without MTZ treatment indicating the cone pathway was not ablated and intact. This scotopic light-off VMR was then used to screen drugs that might improve rod response with the Q344X zebrafish model.

A prominent theory about RP pathogenesis is oxidative stress. Punzo et al., Loss of daylight vision in retinal degeneration: Are oxidative stress and metabolic dysregulation to blame?, J. Biol. Chem. 287:1642-1648 (2012). Since attenuating such stress might slow or prevent RP progression, an ENZO SCREEN-WELL REDOX library was screened against the Q344X zebrafish model. Drug treatment began at 5 dpf to find drugs that can ameliorate the attenuated Q344X scotopic light-off VMR because rod degeneration in this model begins at this stage.

5 dpf larvae were exposed to compounds in this library dissolved in E3 media at a final concentration of 10 μM, and their scotopic light-off VMR was tested at 7 dpf. The drugs of the library came dissolved in dimethyl sulfoxide (DMSO), thus all control larvae were treated with a matching concentration of 0.1% DMSO. All larvae were maintained in the same drug solution throughout the experiment. Each drug was tested twice using embryos collected on different dates.

Of the 84 drugs tested, 16 were toxic to the zebrafish at 10 μM. The VMR of the remaining 68 drug-treated larval groups was normalized and ranked based on the following selection criteria: First, the two biological replicates had to be consistent (consistency determined by a High-Dimensional Nonparametric Multivariate Test between the replicates). A small p value indicating the replicates were dissimilar, whereas a high p value indicative that the replicates were similar. A cut off p value of 0.9 was chosen to select those replicates that were highly similar to each other. Second, the drug-treated VMR had to be significantly different from the DMSO-treated VMR, as determined by the Hotelling's T-squared test. These criteria were applied to two timeframes: 1 second after light offset to capture immediate response, and from 1 to 30 seconds after light offset to capture changes in any of the components of the VMR (Table 1).

TABLE 1
Summary of drug-screening results.

| | 1 s | 30 s |
|---|---|---|
| | timeframe | timeframe |
| Number of starting drugs in the library | 84 | 84 |
| Number of drugs not toxic | 68 | 68 |
| Number of drugs which induced consistent | 5 | 4 |
| light-off scotopic VMR in both replicates | | |
| Number of drugs which induced consistent | 0 | 1 |
| light-off scotopic VMR in both replicates, | | |
| and significantly different from DMSO- | | |
| treated controls (p value < 0.05) | | |

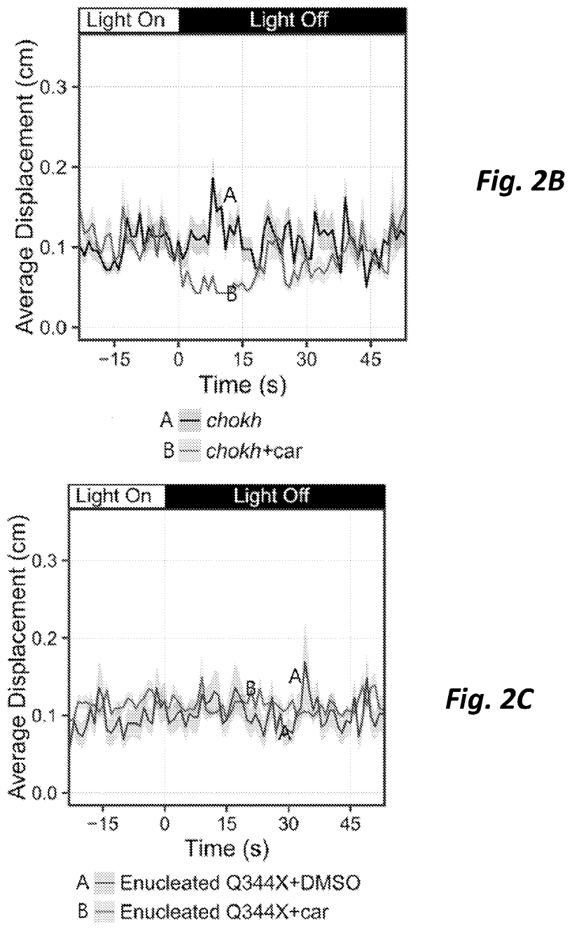

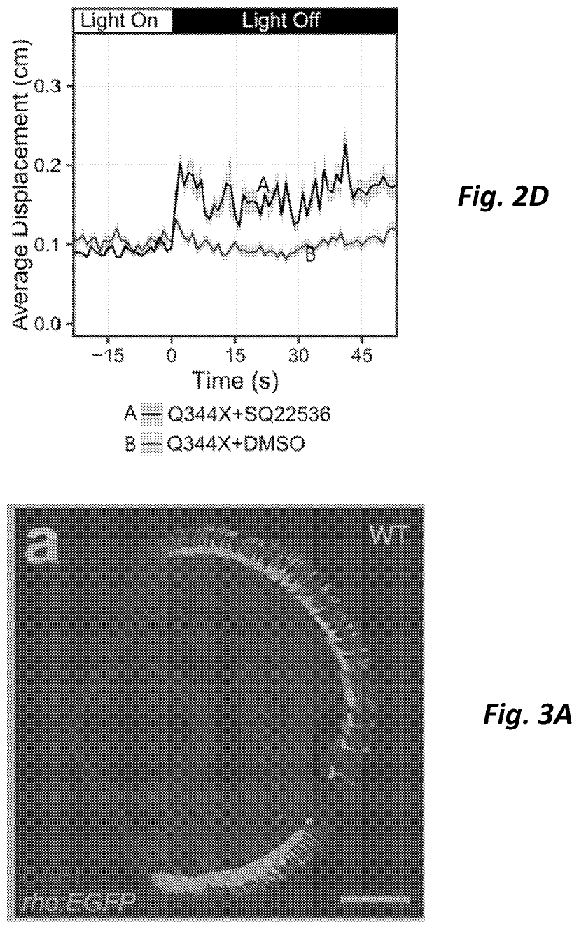

In the 1-second timeframe, 5 drug treatments gave rise to consistent larval behavior, but none of these drug treatments gave rise to a larval VMR that was significantly different from that displayed by DMSO-treated Q344X. However, in the 30-second timeframe, four drug treatments gave rise to a consistent larval behavior, and one drug treatment, carvedilol, provided both a consistent and significant change from the DMSO-treated Q344X VMR. Carvedilol-treated Q344X exhibited a sustained scotopic light-off VMR compared with DMSO-treated WT and Q344X controls (FIG. 2A).

To determine if carvedilol was working through the retina, eyeless chokh/rx3 zebrafish were treated with carvedilol and their VMR was assessed. The chokh/rx3 larvae did not display a light-off VMR with or without carvedilol (FIG. 2B).

Similarly, Q344X larvae were treated with carvedilol or DMSO at 5 dpf and were enucleated at 6 dpf to determine if carvedilol was exerting an effect on extraocular photoreceptors. Neither carvedilol-treated nor DMSO-treated enucleated Q344X larvae displayed a significant scotopic VMR (FIG. 2C). These results suggest that carvedilol works at the level of the retina.

Previous work with the Q344X line has shown that treatment with the ADCY inhibitor SQ 22,536 at a concentration of 100 μM improved rod survival.

To determine if this rod survival translates into improved vision, the Q344X larvae were treated with 100 μM SQ 22,536 from 3 to 7 dpf at a concentration of 100 μM, and their scotopic light-off VMR was assessed at 7 dpf. The ADCY inhibitor was able to produce a significant Q344X VMR (FIG. 2D), however, this response was smaller than that produced by carvedilol treatment.

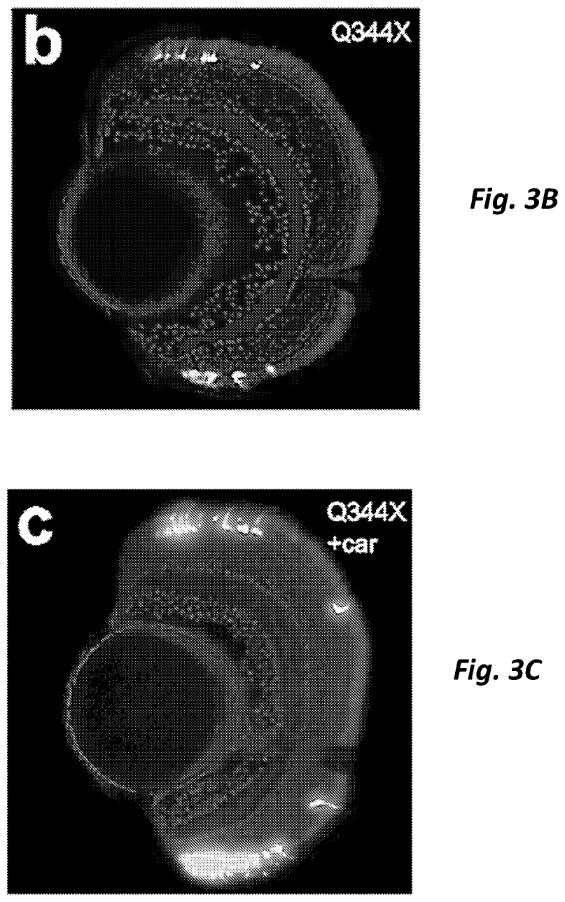

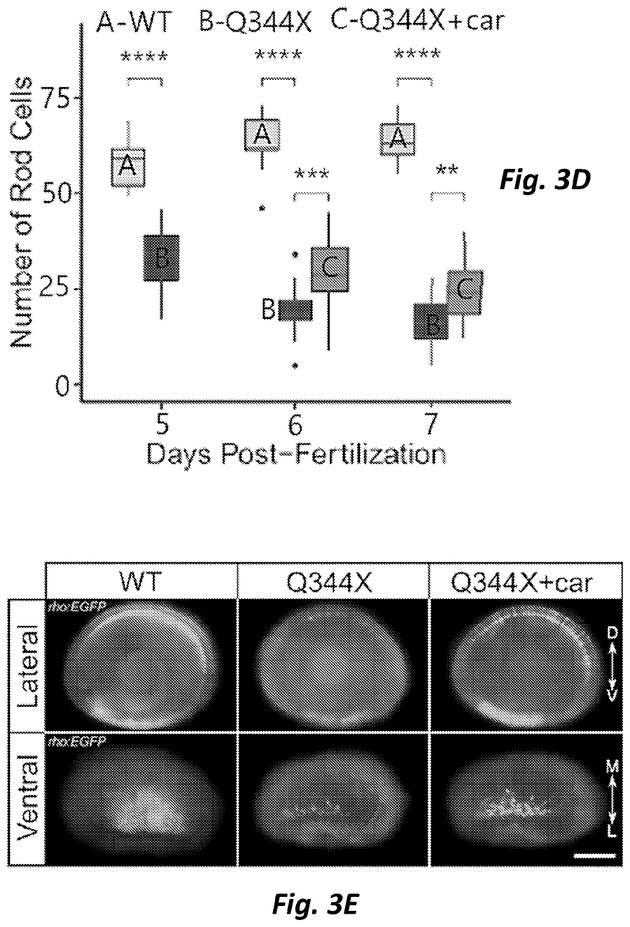

Since carvedilol enhanced the scotopic VMR of the Q344X larvae and acted through the retina, the drug effect on rods was evaluated by quantification of rho: EGFP-positive cells on wholemount and sectioned retinae (FIG. 3A-3D). On cryosections, Q344X larvae exhibited significant rod degeneration on 5 dpf at which point they were treated with carvedilol. Carvedilol-treated Q344X larvae show increased rod number in the retina compared to DMSO-treated Q344X larvae on 6 dpf and 7 dpf (FIG. 3A-3D).

To determine the anatomical distribution of the increased number of rods in the Q344X retina, whole-mount retinae were imaged to assess rod distribution. WT larvae had a high density of rods in the dorsal retina and ventral patch on 7 dpf while Q344X exhibited excessive rod degeneration in these areas (FIG. 3E). Carvedilol-treated Q344X showed an increased number of rods in both the dorsal retina and the ventral patch. To quantify these observations, WT, Q344X, and carvedilol-treated Q344X were binned into three classifications based on the distribution of EGFP signal: Strong, Intermediate, and Weak (Table 2).

TABLE 2
Rod analysis on whole-mount eyes.

| | Strong | Intermediate | Weak |
|---|---|---|---|
| WT lateral | 10 | 0 | 0 |
| Q344X lateral | 0 | 9 | 15 |
| Q344X + car lateral | 0 | 16 | 8 |
| WT ventral | 10 | 0 | 0 |
| Q344X ventral | 0 | 8 | 16 |
| Q344X + car ventral | 0 | 16 | 8 |

All WT larvae were classified as Strong. The carvedilol-treated Q344X larvae had significantly more Intermediate phenotypes in the lateral and ventral views compared to the DMSO-treated Q344X group. No larvae from the carvedilol or DMSO-treated Q344X groups was classified as Strong. The correlation between rod number increase and enhanced light-off VMR of Q344X larvae supported that the increase in rod number with carvedilol treatment mediated the visual improvement.

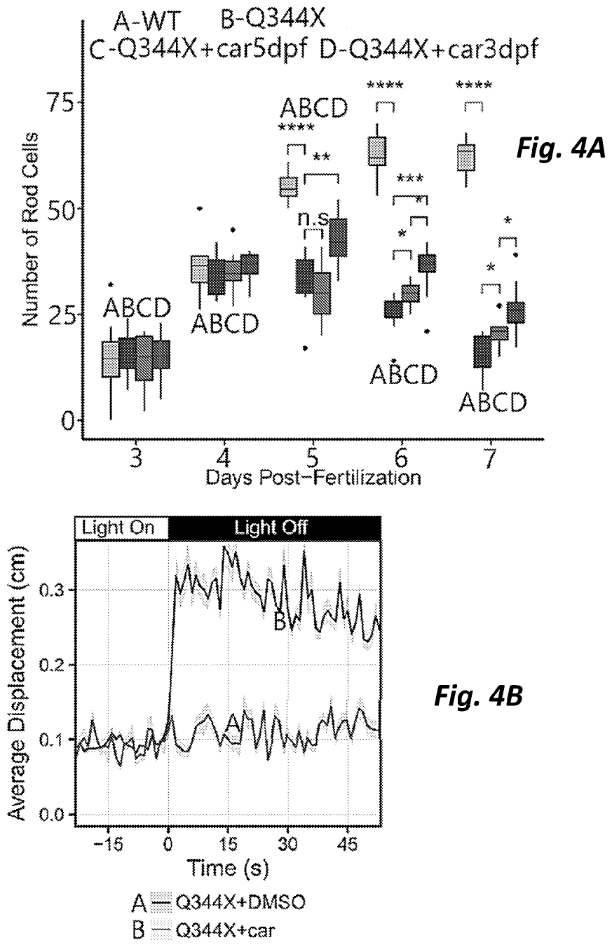

Higher doses of carvedilol were tested at 31.6 μM and 100 μM to determine if a larger treatment dose would improve rod number, but these concentrations were toxic to the zebrafish larvae. Thus, further rod number improvement was evaluated with a longer carvedilol treatment period. Q344X larvae were treated with 10 μM carvedilol beginning at 3 dpf. The drug and media were refreshed daily to maintain the health of the larvae. Larval treatment beginning at 3 dpf was compared to treatment beginning at 5 dpf to determine if earlier carvedilol treatment is more effective. There was no difference in rod number between any of the Q344X and WT groups at 3 dpf and 4 dpf indicating that Q344X rod degeneration is not significant at these stages (FIG. 4A). Q344X rod degeneration does become significant at 5 dpf, and the earlier carvedilol treatment beginning at 3 dpf significantly increased the rod number at 5 dpf (FIG. 4A). Carvedilol treatment beginning at 5 dpf with daily refreshment still improved rod number in the Q344X zebrafish at 6 dpf and 7 dpf, however carvedilol treatment beginning at 3 dpf resulted in significantly higher rod numbers than the later 5 dpf treatment (FIG. 4A). Correlating with increased rod number, the VMR of Q344X larvae treated with carvedilol beginning at 3 dpf displayed a significantly more rapid light-off VMR (FIG. 4B) as compared to the VMR of larvae treated with carvedilol treatment at 5 dpf (Hotellings T-squared test, N=3 replicates of 24 larvae, T=397, df=30, p value <0.0001). Carvedilol treatment beginning at 3 dpf did not have a significant effect on the photopic VMR of Q344X larvae. These results support that earlier carvedilol drug treatment improves the number of Q344X rods better than later treatment, and that the carvedilol effect primarily acts on the rod photoreceptors.

Carvedilol has several known modes of action. It is primarily classified as a β-blocker that binds to β1-adrenergic receptors, β2-adrenergic receptors, α1-adrenergic receptors and inhibits adrenergic signaling. Traditionally. β-blockers were seen only as antagonists that prevent epinephrine from the binding β-adrenergic receptors. Epinephrine can present, for example, in the mouse subretinal space and increase with light exposure. Blocking epinephrine signaling can potentially lower cAMP levels in the Q344X rods by preventing endogenous ADCY signaling. Carvedilol can also act as an atypical β-blocker that is capable of inducing biased signaling. Specifically, carvedilol can promote β-arrestin signaling while acting as an inverse agonist towards G protein signaling. This type of β-arrestin signaling has been shown to have anti-apoptotic effects may prevent Q344X rod death. While carvedilol and other β-blockers have shown to have some beneficial effects in treating other eye-disease models, it was unknown if β-blockers such as carvedilol can work directly on rods.

To evaluate if carvedilol acts directly on rods, the effect of carvedilol treatment on the Y79 human retinoblastoma line was examined, which uniquely expresses rod-specific genes.

The Y79 cell line exists as a photoreceptor-like precursor that shows differentiation potential for the rod lineage. Activin treatment of the Y79 line increases the expression of the transcription factor Nrl which induces progenitor differentiation into rods. Previous work has leveraged this line to conduct expression studies in a photoreceptor-like cellular environment biased towards the rod lineage. The level of adrenergic signaling was determined by GPCR-modulated changes in cAMP levels as measured by a cAMP-sensitive luciferase.

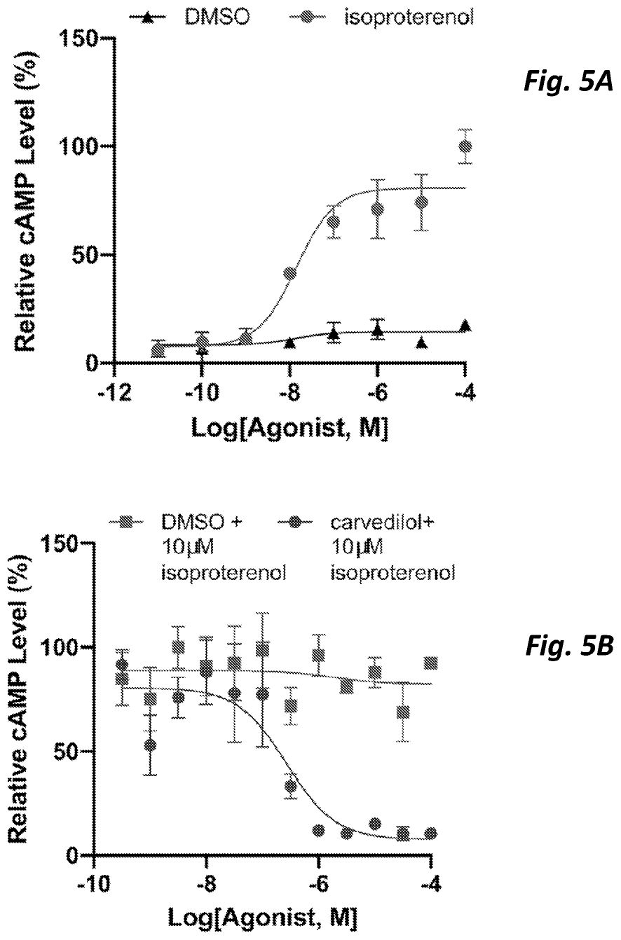

First, the Y79 cells were transfected with the luciferase reporter, and then they were exposed to half-log dilutions of isoproterenol, a β-adrenergic receptor agonist. Isoproterenol was capable of inducing cAMP signaling in the transfected Y79 cells with a pEC50 of 7.5±1.1 (FIG. 5A). The CAMP level was not increased in controls treated with matching DMSO percentage to dissolve isoproterenol. The relative cAMP level did not increase much above 10 μM isoproterenol.

To determine if carvedilol treatment can inhibit this isoproterenol-mediated cAMP increase, the transfected Y79 cells were pretreated with half-log dilutions of carvedilol and then challenged with a dose of 10 μM isoproterenol that would induce saturating relative cAMP level according to FIG. 5A. Carvedilol pretreatment was able to prevent isoproterenol-mediated cAMP signaling with a pIC50 of 6.5±0.7 (FIG. 5B).

### Example 2

### In Vivo Rod Development Assay

Transgenic zebrafish Gmc500 (Tg(rho: YFP-NTR)) in which rod photoreceptors expressed yellow fluorescence protein (YFP) and nitroreductase (NTR) were bred for embryo collection. The embryos were routinely raised in E3 media at 28° C. and treated with propylthiouracil (PTU) starting from 16 hpf to suppress the black pigmentation and facilitate YFP quantification.

Larvae fish were transferred to 96-well plates and exposed to 10 μM carvedilol (CAR) from 3-7 dpf or 3-5 dpf as described above in Example 1. An in vivo visual-behavioral screen was then employed to quantify each larva's VMR as described in Example 1.

Rod-YFP levels were quantified at 7 dpf using a published method ARQiv (automated reporter quantification in vivo). See Walker et al., Automated reporter quantification in vivo: high-throughput screening method for reporter-based assays in zebrafish. PLoS ONE 7: E29916 (2012) and White et al., ARQiv-HTS, a versatile whole-organism screening platform enabling in vivo drug discovery at high-throughput rates, Nat. Protoc. 11:2432-2453 (2016). Retinoic acid served as a positive control drug and each assay was conducted in triplicates. To analyze the result, all readings were first normalized by the average reading of the same-day DMSO control fish. Data from three different experimental days were pooled for statistical analysis. Student t-test was performed followed by Bonferroni correction. The adjusted alpha cutoff was 0.05/3=0.0167.

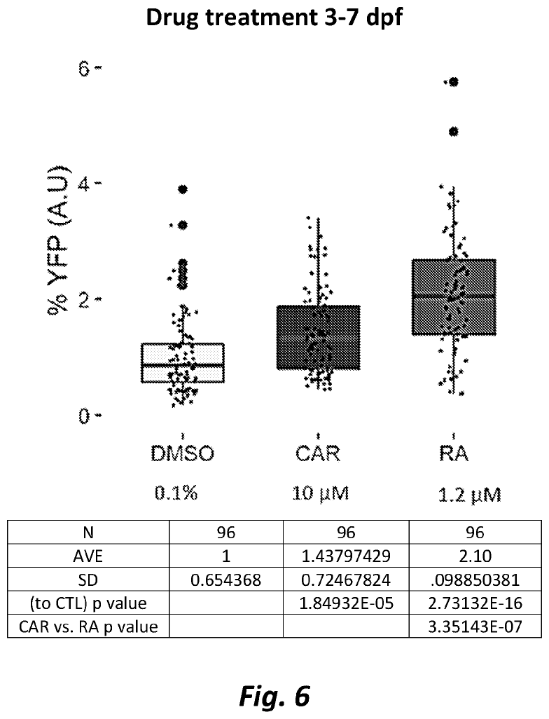

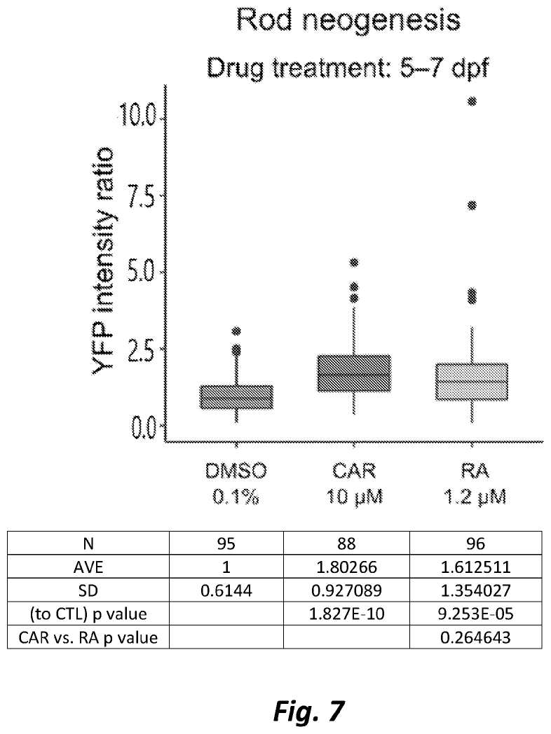

FIGS. 6 and 7 show the results of both CAR treatment 3-7 dpf and CAR treatment 5-7 dpf. The results support that CAR can significantly facilitate rod genesis during early retina development in both treatment schemes, with both CAR treatment groups indicating additional rod development as compared to the control group (DMSO). Compared to the RA positive control, CAR was less effective in the 4-day treatment (3-7 dpf), but more effective in the 2-day treatment (5-7 dpf).

### Example 3

### Rod Regeneration Assay

Transgenic larvae fish (described above) were treated with the prodrug metronidazole (MTZ) at 5 dpf to ablate rod photoreceptors for 24 hours, rinsed with E3/PTU at 6 dpf and then exposed to either 10 μM carvedilol (CAR treatment group), 0.1% DMSO (ablated control group) 2.5 μM prednisolone (PRE), or 2.5 μM dexamethasone (DEX), each in 96-well plates with 1 fish per well. Rod-YFP levels were quantified at 9 dpf. PRE and DEX were used as positive controls and the assay was one biological repeat. All readings were first normalized by the average reading of the same-day DMSO control fish without MTZ ablation or any drug treatment ((−) 0.1% DMSO control group). Student t test was performed followed by Bonferroni correction. The adjusted alpha cutoff was 0.05/4=0.0125.

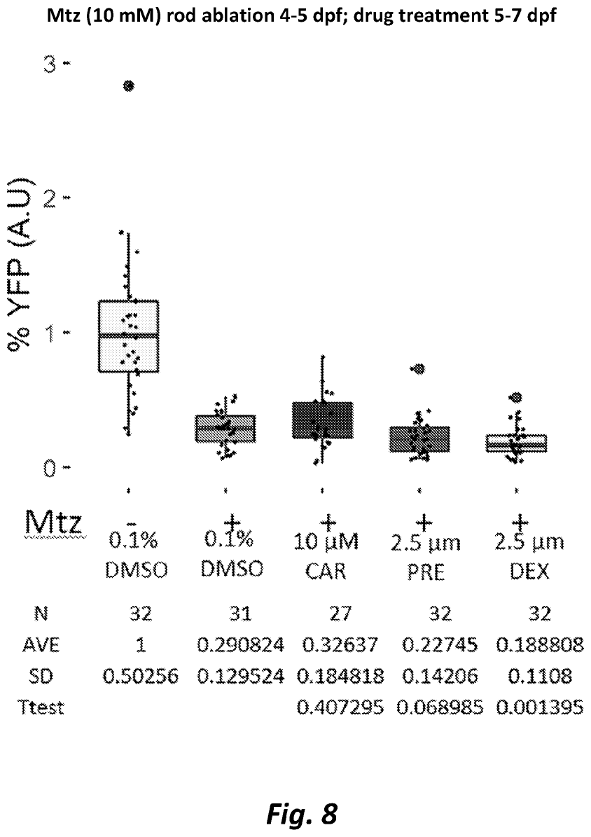

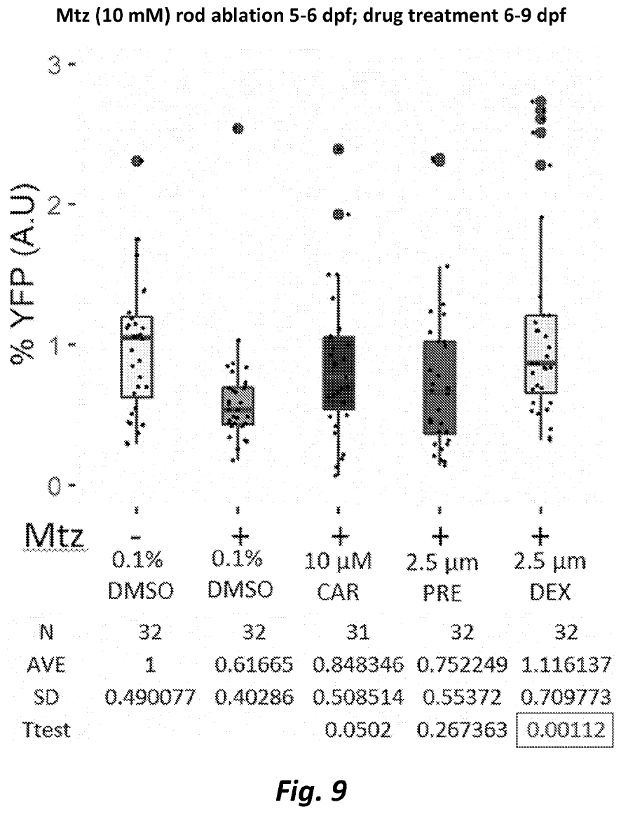

FIG. 8 shows the results of MTZ rod ablation 4-5 dpf, with drug treatment 5-7 dpf. FIG. 9 shows the results of MTZ rod ablation 5-6 dpf and drug treatment 6-9 dpf. The data supports CAR had no significant effect on rod regeneration in the MTZ-induced rod cell death model. In the 6-9 dpf treatment model more rods were observed in the CAR-treated fish as compared to the control groups (DMSO, PRE and DEX), and rods regenerated back to about 60% of the nonablated controls (FIG. 9). In the 5-7 dpf treatment (FIG. 8), significant rod regeneration was not observed (about 30% of the nonablated controls). The positive control group DEX showed significant regenerative effect, while the positive control group PRE did not.

### Example 4

### Selleckchem Drug Library Screening Study

A Selleckchem FDA-approved drug library with 1430 compounds was screened against the Q344X zebrafish model. The drug screen was conducted as described in the previous drug screening study. Briefly, drug treatment began at 5 dpf to find drugs that can ameliorate the attenuated Q344X scotopic light-off VMR because rod degeneration in this model begins at this stage.

5 dpf larvae were exposed to compounds in this library dissolved in E3 media at a final concentration of 10 μM, and their scotopic light-off VMR was tested at 7 dpf. The drugs of the library came dissolved in DMSO, thus all control larvae were treated with a matching concentration of 0.1% DMSO. All larvae were maintained in the same drug solution throughout the experiment. Each drug was tested twice using embryos collected on different dates.

The VMR of the drug-treated larval groups was normalized and ranked based on the selection criteria described in Example 1 with respect to the previous drug screening study. These criteria were applied to two timeframes: 1 second after light offset to capture immediate response, and from 1 to 30 seconds after light offset to capture changes in any of the components of the VMR.

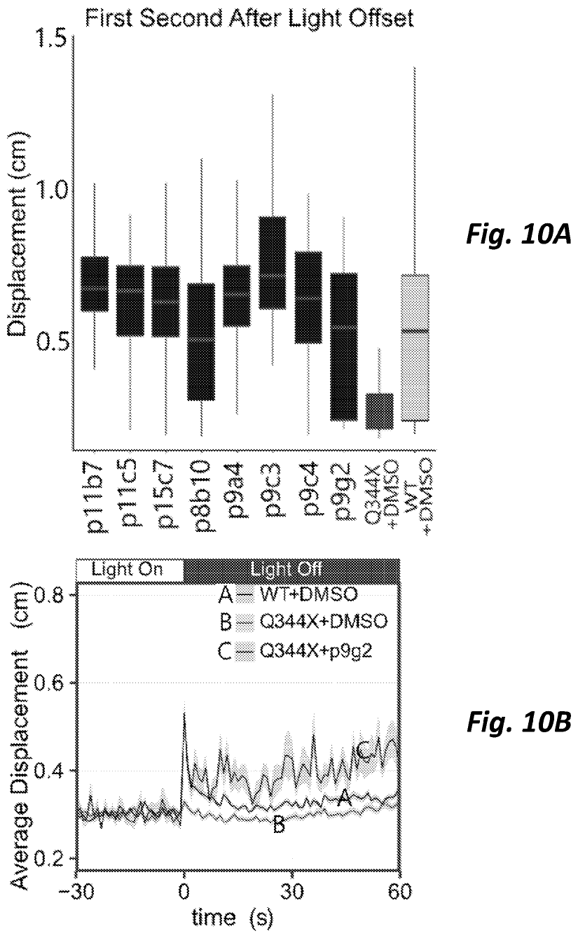

From this screen, eight initial hits—compounds esmolol hydrochloric acid (a cardioselective β-blocker) (p11b7), triamterene (a transmembrane transporter than can block epithelial Na+ channels (ENaC) in a voltage-dependent manner with IC150 of 4.5 μM) (p11c5), trelagliptin (SYR-472, a protease that can be a highly-selective, long-acting dipeptidyl peptidase-4 inhibitor) (p11c7), prednisolone acetate (Omnipred, a synthetic corticosteroid that can be effective as an immunosuppressant agent to, for example, reduce irritation, redness, burning and swelling) (p8b10), crenolanib (CP-868596: a protein tyrosine kinase) (p9a4), dolutegravir (GSK1349572) (p9c3), tivantinib (ARQ 197, a protein tyrosine kinase) (p9c4), and noradrenaline bitartrate monohydrate (a direct alpha-adrenergic receptor stimulator) (p9g2)—were identified that improved the light-Off VMR of Q344X larvae (FIG. 10A), and it is hypothesized that this improved function is due to increases in the rod number of the Q344X larvae.

To evaluate rod number and morphology, histological analysis is performed on the animals treated with the identified drug hits and compared with controls (as described in the previous examples). The same drug-treatment protocol is used in the drug screening (i.e. 5 to 7 dpf). The Q344X fish line carries a rod reporter transgene that expresses EGFP in rods which facilities the proposed histological analysis.

To identify drug hits that induce a WT-like VMR profile, metrics such as univariate Euclidean distance or area under curves (AUC) are used to quantify and rank the drug effect similar to the WT control. The AUC measures the area under the distance curve (see FIG. 10B). After this analysis, the histological effect of treatment with the identified drug hits is analyzed.

### Example 5

### LOPAC1280 Drug Library Screening Study

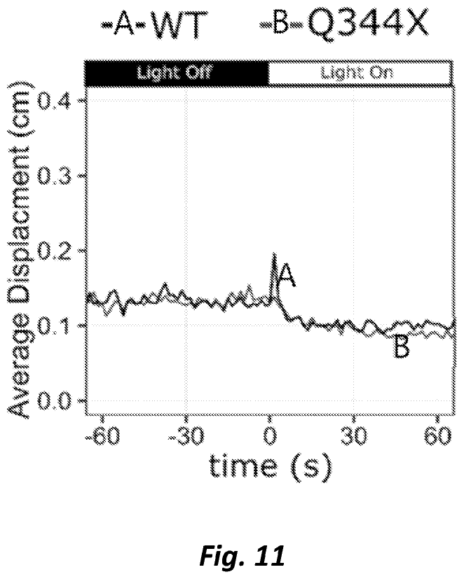

A LOPAC1280 drug library is screened against the Q344X zebrafish model, using the optimized light-On (FIG. 11) and light-Off (FIGS. 9, 10A, and 10B) VMR for rod response. The LOPAC1280 library contains pharmacologically active compounds targeting cellular processes such as neurotransmission, apoptosis, and G-protein coupled receptor signaling. This library has been successfully used in zebrafish to identify drugs for promoting oligodendrocyte migration (Fontenas et al., The neuromodulator adenosine regulates oligodendrocyte migration at motor exit point transition zones, Cell Rep 27:115-128 e5 (2019)), inducing pigment cell differentiation (Camargo-Sosa et al., Endothelin receptor Aa regulates proliferation and differentiation of Erb-dependent pigment progenitors in zebrafish, PLoS Genet 15: e1007941 (2019)), and repressing pathologic angiogenesis (Tran et al., Automated, quantitative screening assay for antiangiogenic compounds using transgenic zebrafish, Cancer Res 67:11386-11392 (2007)). Screening this established LOPAC1280 library with two distinct light stimuli enhances the chances of identifying additional positive hits for treating Q344X adRP.

Potentially toxic compounds are eliminated by treating the Q344X larvae with the individual compounds in the library at 10 μm from 5 dpf to 7 dpf (the treatment scheme used in the above-described studies (see Ganzen et al., 2021, supra), with the remaining non-toxic compounds are used for the actual screening.

To maximize the screening efficiency, an orthogonal pooling strategy is used. Ohnesorge et al., Orthogonal drug pooling enhances phenotype-based discovery of ocular antiangiogenic drugs in zebrafish larvae, Front Pharmacol 10:508 (2019). This strategy combines compounds in a row or in a column in 96-well or 384-well plates as a compound pool for VMR assay. This orthogonal-pooling strategy can likely substantially reduce the time for screening to one year. In each prepared pool, each single compound is diluted at 10 μm and the total DMSO carrier will be 1%. Each pool is applied to 24 individual larvae. Drug-carrier treated Q344X and WT larvae are used for negative and positive controls, respectively. The VMR data is analyzed with our established statistical methods to identify positive hits. See. e.g., Liu et al., Statistical analysis of zebrafish locomotor response, PLoS ONE 10:e30139521 (2015); Liu et al., Statistial analysis of zebrafish locomotor behaviour by generalized linear mixed models, Sei Rep 7(1): 2937 (2017); Xie et al., Normalization of large-scale behavioural data collected from zebrafish, PLoS ONE 14(2); e0212234 (2019). Histology studies (as described above) are then conducted on the positive compound hits to define the effect of positive drugs on rod number and morphology.

The number of expected toxic compounds in the LOPAC1280 library is expected to be around 115 to 244, and the number of non-toxic compounds is expected to be from 1036-1165. Based on the positive rates in the ENZO REDOX library (1/68 or 1.5%) and Selleckchem FDA-approved compound library (8/1301 or 0.6%), the number of positive compounds is expected to be from 6 to 17. These LOPAC positive hits are expected to increase the VMR and increase the number of rods in the treated Q344X larvae compared with the untreated controls. WT-like analysis will also be utilized on the LOPAC dataset.

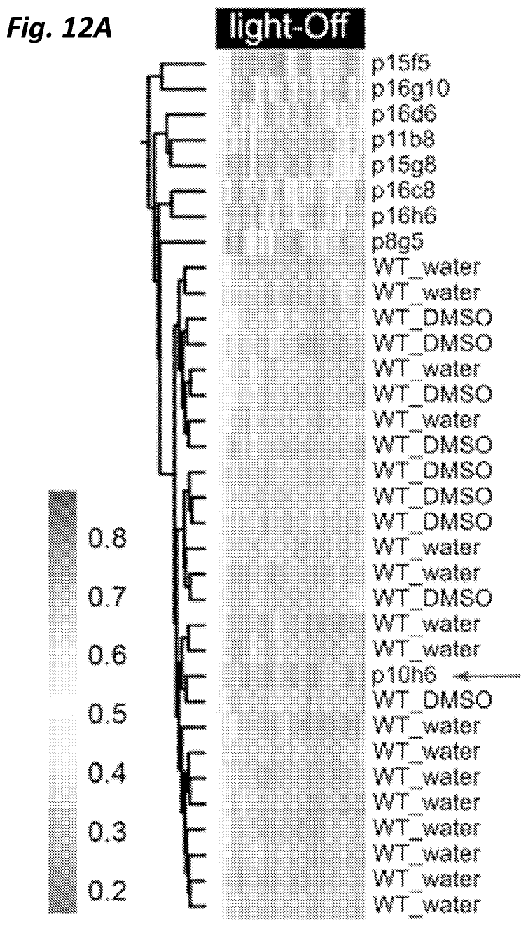

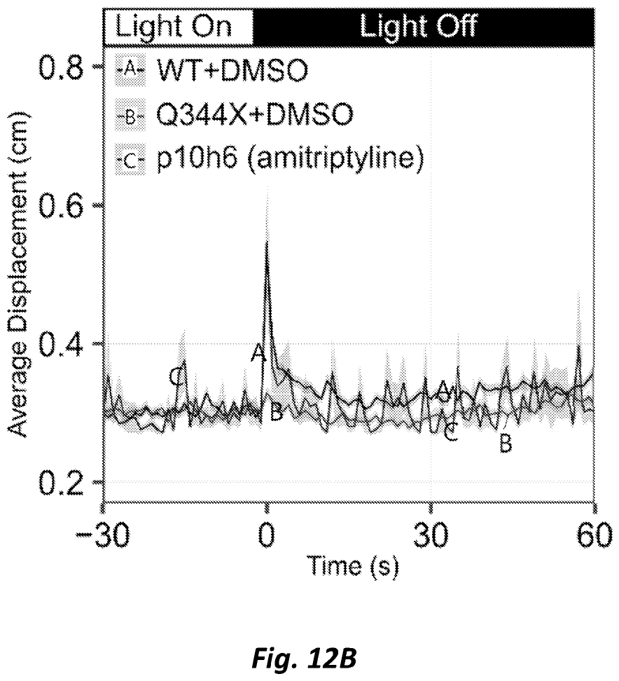

Hierarchical clustering (HC) was also used to identify another 9 hits that induced a WT-like VMR, with 1-30 seconds of the light-Off VMR used to calculate the pairwise Euclidean distance of the displacement values for HC with the complete-linkage method. All WT controls treated with drug carriers clustered tightly with 9 hits that have activities on cell cycle, DNA damage, metabolism, and neural signaling, which may indicate a curative effect (FIG. 12A). The clade was significant, based on a bootstrap analysis (p<0.05). Suzuki & Shimodaira, pvclust: An R package for hierarchical clustering with p-values, Bioinformatics 22:1540-1542 (2006). Additionally, the colors in the heatmap showed the displacement values as indicated (with the majority of the ranges in the 0.2-0.04 values).

One of the hits, amitriptyline (p10h6), was clustered tightly with all WT controls (FIG. 12A, arrow) and induced the Q344X mutant to display a WT-like VMR (FIG. 12B). Amitriptyline has been shown to induce neuroprotectants glial cell line-derived neurotrophic factor (GDNF) and pigment epithelium-derived factor (PEGF) in retinal cells. Baranov et al., Low-oxygen culture conditions extend the multipotent properties of human retinal progenitor cells, Tissue Engineering Part A 20(9-10): 1465-1475 (2014); Baranov et al., Optimizing the conditions and use of synthetic matrix for three-dimensional in vitro retinal differentiation from mouse pluripotent cells, Tissue Engineering Part C Methods 25(7): 433-445 (2019).

These neuroprotectants can slow down retinal degeneration in several RP models, including a rat model with the RHO S334X mutation, which is a Class I mutation similar to the Q344X. Dalkara et al., AAV mediated GDNF secretion from retinal glia slows down retinal degeneration in a rat model of retinitis pigmentosa, Molecular Therapy 19(9): 1602-1608 (2011); Sanftner et al., Glial cell line derived neurotrophic factor delays photoreceptor degeneration in a transgenic rat model of retinitis pigmentosa, Molecular Therapy 4(6): 622-629 (2001); Cayouette et al., Pigment epithelium-derived factor delays the death of photoreceptors in mouse models of inherited retinal degenerations, Neurobiology of Disease 6(6): 523-532 (1999).

### Example 6

### Drug Hits Can Suppress Rod Death and or Promote Rod Neogenesis

Rod degeneration in RP has been thought to be driven by apoptosis. Portera-Cailliau et al., Apoptotic photoreceptor cell death in mouse models of retinitis pigmentosa, Proceedings National Academy of Sciences USA 91(3): 974-978 (1994); Nakao et al., Intravitreal anti-VEGF therapy blocks inflammatory cell infiltration and re-entry into the circulation in retinal angiogenesis, Retina 53(7): 4323-4328 (2012); Hollingsworth et al., DPP9 sequesters the C terminus of NLRP1 to repress inflammasome activation, Nature 592:778-783 (2021). In the Q344X adRP, the mutated RHOs are mis-trafficked to the plasma membrane and are still functional. Hollingsworth & Gross, Defective trafficking of rhodopsin and its role in the retinal degenerations, Internat'l Review Cell & Molecular Biology 293:1-44 (2012); Concepcion & Chen, Q344ter mutation causes mislocation of rhodopsin molecules that are catalytically active: a mouse model of Q344ter-induced retinal degeneration, PLOS One 5 (6); e10904 (2010). They can aberrantly activate ADCY and, in turn, apoptosis. Nakao et al. (2012), supra. In the present investigator's REDOX screen, it was identified that CAR enhanced the VMR of the Q344X mutant and increased its rod numbers. Ganzen et al., 2021, supra.

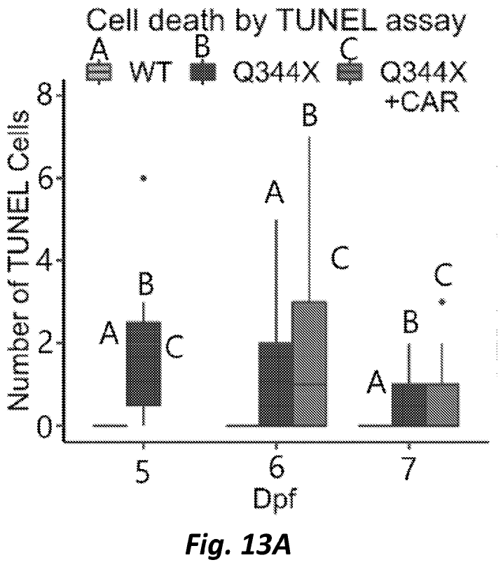

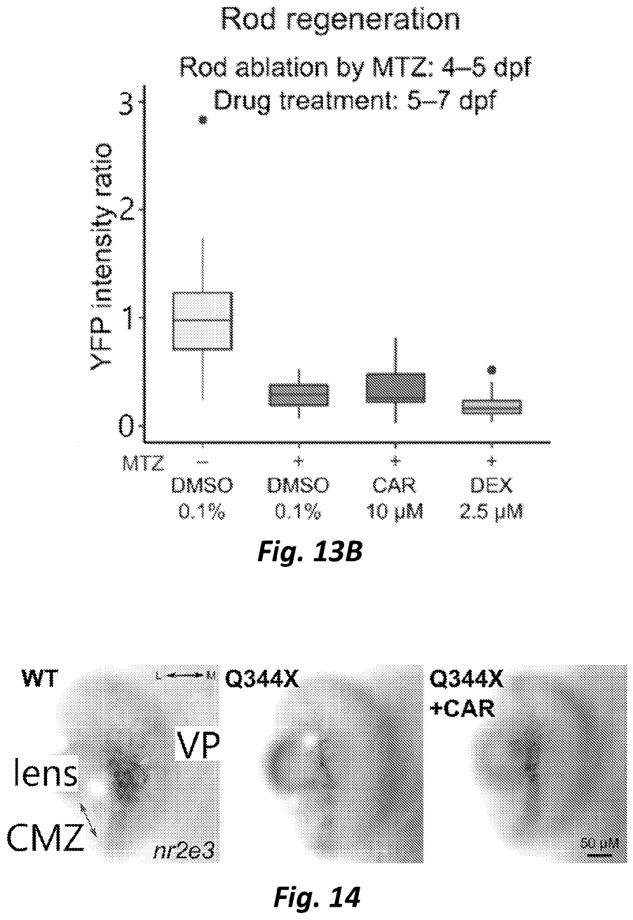

Terminal deoxynucleotidyl transferase-mediated dUTP nick end labeling (TUNEL) assay's were performed (using protocols well-known in the art), which is a commonly used approach for detecting apoptosis. Surprisingly, a substantial change in cell death was not detected by the TUNEL assay (FIG. 13). Compared to WT, the Q344X retinas had more TUNEL-positive cells at 5 dpf (WT, n=11; Q344X, n=19; Welch's Two Sample t-test, p=9.8e-05), at 6 dpf (WT, n=9; Q344X, n=25; Welch's Two Sample 1-test, p=0.0003), and at 7 dpf (WT, n=8; Q344X, n=22; Welch's Two Sample t-test, p=0.002). The CAR treatment beginning on day 5 dpf did not alter the number of TUNEL-positive cells at 6dpf (Q344X, n=25; CAR, 6dpf n=24; Welch's Two Sample 1-test, p=0.36), or 7dpf (Q344X, n=22, CAR, n=14; Welch's Two Sample t-test, p=0.81). This data suggests that CAR likely did not exert its positive effect on the Q344X mutant by reducing apoptotic cell death. However, the TUNEL assay is not specific to apoptotic cells; it can also label necroptotic cells. Kraupp et al., In situ detection of fragmented dna (tunel assay) fails to discriminate among apoptosis, necrosis, and autolytic cell death: a cautionary note, Hepatology 21(5): 1465-1468 (1995). Rod degeneration in RP can also be triggered by several cell-death pathways, including caspase-independent pathway, necroptosis, and parthanatos. Viringipurampeer et al., Rip3 knockdown rescues photoreceptor cell death in blind pde6c zebrafish, Cell Death & Differentiation 21(11): 1320-1329 (2014); Arango-Gonzalez et al., Identification of a common non-apoptotic cell death mechanism in hereditary retinal degeneration, PLOS One 9(11): e112142 (2014); Sanvicens et al., Oxidative stress-induced apopotosis in retinal photoreceptor cells is mediated by calpains and caspases and blocked by the oxygen radical scavenger CR-6, Molecular Basis of Cell & Developmental Biology 279(38): 39268-39278 (2004); Sanges et al., Apoptosis in retinal degeneration involves cross-talk between apoptosis-inducing factor (AIF) and caspase-12 and is blocked by calpain inhibitors, Biological Sciences 103(46): 17366-17371 (2006); Zhang et al., Emerging role of exosomes in retinal diseases, Frontiers Cell Developmental Biology 9:643690 (2021); Newton & Megaw, Mechanisms of photoreceptor death in retinitis pigmentosa, Genes 11(10): 1120 (2020).

In the retinas of the mouse Q344X model, the proinflammatory pathways are activated, a hallmark more consistent with non-apoptotic pathways including necroptosis. Hollingsworth et al. (2021), supra; Pasparakis & Vandenabeele. Necroptosis and its role in inflammation, Nature 517 (7534:311-320 (2015). Several cell-death pathways can even be simultaneously initiated by one RHO S334X mutation, a Class I mutation which is very similar to the Q344X mutation. Arango-Gonzalez et al. (2014), supra. Therefore, these pathways could mediate rod degeneration in the Q344X mutant and provide new targets for the drug hits to act on. To that end, the extent to which these pathways drive Q344X rod death was studied before characterizing how the drug hits prevent Q344X rod death.

Another possible explanation of CAR's effect on the Q344X mutant is that CAR promoted the generation of new rods, either through rod neogenesis or regeneration. Rod neogenesis is likely, as the extra rods in the CAR-treated group were mostly detected close to CMZ, a circular zone on the lateral edge of the retina. The CMZ generates retinal neurons continuously in zebrafish after the first wave of neurogenesis at 3 dpf. Fadool & Dowling. Zebrafish: a model system for the study of eye genetics, Progress in Retinal & Eye Research 27(1): 89-110 (2007). It generates new rods through seeding stem cells called Müller cells (MCs) in the inner nuclear layer. Nelson et al., The developmental sequence of gene expression within the rod photoreceptor lineage in embryonic zebrafish. Developmental Dynamics 237:2903-17 (2008); Bernardos et al., Late-stage neuronal progenitors in the retina are radial Müller glia that function as retinal stem cells. J Neuroscience 27(26): 7028-7040 (2007); Otteson et al., Putative stem cells and the lineage of rod photoreceptors in the mature retina of the goldfish, Developmental Biology 232(1): 62-76 (2001). These stem cells generate new rods, as early as 60 hours post-fertilization (hpf). Even though zebrafish can regenerate their retina, the photoreceptor takes a longer time to regenerate than the drug-treatment scheme described herein. Walker et al. (2012), supra: Fraser et al., Regeneration of cone photoreceptors when cell ablation is primarily restricted to a particular cone subtype, PLOS One 8(1): e55410 (2013); Yoshimatsu et al., Presynaptic partner selection during retinal circuit reassembly varies with timing of neuronal regeneration in vivo, Nature Communications 7:10590 (2016). Therefore, regeneration is less likely the mechanism that CAR activated in the Q344X retina. Nonetheless, the extent to which CAR affects rod neogenesis (FIG. 7) and regeneration (FIG. 8) was determined by a fluorescence-based approach described in Zhang et al. (2021), supra.

Briefly, in the rod-neogenesis assay (FIG. 7), a WT rod-reporter line Tg(rho: YFP-NTR) was exposed from 5 to 7 dpf to: (1) DMSO (control), (2) 10 μM CAR, and (3) 1.2 μM retinoic acid (RA) (+ve control) that was known to promote rod fates for the same period. Granzen et al. (2021), supra; Hyatt et al., Retinoic acid alters photoreceptor development in vivo, Neuroscience 92(23): 13298-13303 (1996). The resulting fluorescence was measured on a scanner. Both the CAR and RA groups significantly increased the YFP signal in the larvae as compared with DMSO-treated controls, supporting that CAR likely promoted rod neogenesis (FIG. 7). This is also supported by an expansion of a rod-specification marker nr2e3 proximal to CMZ in the CAR-treated Q344X retinas (FIG. 14). Nelson et al. (2008), supra; Chen et al., The rod photoreceptor-specific nuclear receptor nr2e3 represses transcription of multiple cone-specific genes, J Neuroscience 25(1): 118-129 (2005).

Since the extra rods in the CAR-treated larvae still contained the Q344X transgene that continuously triggered cell death, these rods might not function normally and gave rise to the observed VMR. In the rod-regeneration assay (FIG. 8), the Tg(rho: YFP-NTR) line was first treated from 4 to 5 dpf with metronidazole (MTZ), a prodrug that would be catalyzed into cytotoxic substance in the presence of bacterial nitroreductase (NTR). Since NTR was located in the construct rho: YFP-NTR, the larval rods would be ablated by MTZ incubation from 4 to 5 dpf (FIG. 8. MTZ (−) vs (+)). Then, these rod-ablated larvae were exposed from 5 to 7 dpf to (1) DMSO; (2) 10 μM CAR; or (3) 2.5 μM dexamethasone (DEX), which is known to promote rod regeneration. White et al., Immunomodulation-accelerated neuronal regeneration following selective rod photoreceptor cell ablation in the zebrafish retina, Proc Nat'l Acad Science 114: E3719-E3728 (2017). The resulting fluorescence was measured on a scanner. Neither treatment increased the YFP signal as compared to the rod-ablated DMSO controls (FIG. 8); in fact, the DEX treatment actually decreased the YFP signal. These results suggest that, during the experimental drug-treatment period (i.e., 5 to 7 dpf), CAR increased the rod number in the Q344X mutant by rod neogenesis and not by promoting regeneration or suppressing cell death. Even though CAR may not be a specific treatment for Q344X adRP, its rod-neogenesis property can be harnessed to generate new rods from WT stem cells for research and cell-based therapy to treat a broader range of RP subtypes.

### CERTAIN DEFINITIONS

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

1. A method for photoreceptor rod regeneration or rod genesis comprising administering, to a subject, a therapeutically effective dose of therapeutic agent selected from the group consisting of carvedilol, esmolol hydrochloric acid, triamterene, trelagliptin, prednisolone acetate, crenolanib, dolutegravir, tivantinib, noradrenaline bitartrate monohydrate, vidofludimus, gabapentin, gemcitabine hydrochloride (HCl), desvenlafaxine succinate, LCZ696 or sacubitril/valsartan, Palbociclib (PD0332991) isethionate, galanthamine hydrobromide (HBr), amitriptyline HCl, and xylazine HCl.

2. The method of claim 1, wherein the subject has enhanced regeneration or genesis of photoreceptor rods as compared to a control subject that did not receive administration of the therapeutic agent.

3. The method of claim 1, wherein the therapeutic agent is carvedilol.

4. The method of claim 3, wherein the therapeutically effective dose of carvedilol is administered to the subject before onset of a retinal-degenerative disease and/or comprises at least two therapeutically effective doses administered to the subject over a period of at least two days.

5. (canceled)

6. The method of claim 1, wherein the therapeutically effective dose is administered to an eye of the subject.

7. The method of claim 1, wherein the therapeutically effective dose is administered in a manner that facilitates delivery of the therapeutically effective dose to a retina of the eye of the subject.

8. The method of claim 1, wherein the therapeutically effective dose is administered topically.

9. The method of claim 6, wherein the therapeutically effective dose is formulated as topical eye drops.

10. The method of claim 1, wherein the method is for rod genesis and the therapeutically effective dose is administered to the subject during early retinal development, or the method is for rod regeneration and the therapeutically effective dose is administered to a mature subject.

11. The method of claim 1, wherein the method is for rod regeneration and the therapeutically effective dose is administered to a mature subject.

12. The method of claim 1, wherein the subject is experiencing, or at risk of experiencing, a retinal-degenerative disease.

13. The method of claim 12, wherein the retinal-degenerative disease is retinitis pigmentosa.

14. The method of claim 1, further comprising administering at least one additional therapeutic, pharmaceutical, biochemical, or biological agent or compound to the subject.

15. The method of claim 14, wherein the at least one additional therapeutic, pharmaceutical, biochemical, or biological agent or compounds is administered to the eye of the subject.

16-22. (canceled)

23. A method for the in vitro production of a population of rod photoreceptors comprising culturing retinal progenitor cells under conditions and for a period of time that enable cell growth and differentiation of the retinal progenitor cells to produce photoreceptor progenitor cells, wherein the conditions comprise exposure to a therapeutically effective dose of a therapeutic agent selected from the group consisting of carvedilol, esmolol hydrochloric acid, triamterene, trelagliptin, prednisolone acetate, crenolanib, dolutegravir, tivantinib, noradrenaline bitartrate monohydrate, vidofludimus, gabapentin, gemcitabine hydrochloride (HCl), desvenlafaxine succinate, LCZ696 or sacubitril/valsartan, Palbociclib (PD0332991) isethionate, galanthamine hydrobromide (HBr), amitriptyline HCl, and xylazine HCl.

24. The method of claim 23, further comprising culturing pluripotent stem cells to produce one or more retinal progenitor cells.

25. The method of claim 23, further comprising dissociating an extracellular matrix of retinal tissue or retinal tissue fragments from a subject so to dissociate retinal progenitor cells from each other without lysing the retinal progenitor cells.

26. The method of claim 25, wherein the retinal tissue or retinal tissue fragments are mammalian retinal tissue or mammalian retinal tissue fragments.

27. A population of photoreceptor progenitor cells or photoreceptor rods generated therefrom, wherein the photoreceptor progenitor cells are obtained by the method of claim 23.

28-30. (canceled)
